(19) United States
(12) Patent Application Publication
(10) Pub. No.: US 2026/0216149 A1
(43) Pub. Date: Jul. 30, 2026

(54) USE OF DIAZAPHENANTHRENE PROTONATED DIMER

(71) Applicant: XI’AN LIZE TECHNOLOGY PROJECT INVESTMENT CO., LTD., Xi'an (CN)

(72) Inventors: Zizhen Zhao, Xi'an (CN); Zhigang Wang, Xi'an (CN); Ailing Fu, Chongqing (CN); Chen Fu, Chongqing (CN)

(21) Appl. No.: 19/147,652

(22) PCT Filed: Jan. 11, 2023
(86) PCT No.: PCT/CN2023/071813

§ 371 Date: Jul. 11, 2025

(51) Int. Cl. A61K 31/4375; A61P 35/00; C07D 471/04

(52) U.S. Cl. CPC A61K 31/4375; C07D 471/04; A61P 35/00

(57) ABSTRACT

Provided are a protonated dimer cation of 1,10-phenanthroline or a derivative thereof, and a salt comprising a cation and a first anion. The cation or the salt can be used for preparing an anti-tumor drug or an antimicrobial agent.

### FIELD

The present application relates to the field of biomedicine, and more particularly, to a protonated dimer of 1,10-phenanthroline or a derivative thereof, and use of the protonated dimer, especially use in anti-tumor and antimicrobial aspects, and the like.

### BACKGROUND

In medicine, carcinoma refers to a malignant tumor originated from epithelial tissues and is the most common type of malignant tumors. Correspondingly, malignant tumors originating from mesenchymal tissues are collectively referred to as sarcomas. There are a few malignant tumors that are not named according to the above principles, such as nephroblastoma, and malignant teratoma. The term “cancers” commonly known by people generally refers to all malignant tumors. Cancers have biological characteristics such as abnormal cell differentiation and proliferation, uncontrolled growth, invasion and metastasis, and occur in a multi-factor and multi-step complex process, which is divided into three processes: carcinogenesis, cancer promotion, and evolution, which are closely related to smoking, infections, occupational exposure, environmental pollution, unreasonable diet, and genetic factors.

There are many types of malignant tumors, with different natures, different tissues and organs involved, different periods of onset, and different responses to various treatments. Therefore, most patients require comprehensive treatment. The so-called comprehensive treatment is to use surgery, chemotherapy, radiotherapy, immunotherapy, traditional Chinese medicine treatment, interventional treatment, microwave treatment and other means based on patient's physical conditions, pathological types of tumors, invasion ranges, etc., in order to significantly improve a cure rate and improve the patient's quality of life.

Chemotherapy is an abbreviation for treatment with chemotherapy drugs, which achieves a treatment purpose by using chemotherapy drugs to kill cancer cells. Chemotherapy is one of the most effective treatments for cancers, and together with surgery and radiotherapy, is known as the three major treatments for cancers. Surgery and radiotherapy are local treatments, which are only effective for tumors at a treated site, and are difficult to effectively treat potential metastatic lesions and cancers that have metastasized clinically. Chemotherapy is a form of systemic treatment. Regardless of a route of administration (oral, intravenous, or intracavitary administration, etc.), chemotherapy drugs will spread to most of the organs and tissues throughout the body through the blood circulation. Therefore, for some tumors with a tendency to spread throughout the body and middle and advanced stages of tumors that have metastasized, chemotherapy is the main treatment means.

However, current chemotherapy drugs generally target cells with a proliferative ability throughout the body, resulting in great damages to normal cells in the body while killing tumor cells, a very poor prognosis for patients, as well as hair loss, weight loss, decreased immunity and other adverse effects. Therefore, there is a need to develop chemotherapy drugs that have selective inhibition for tumor cells, without any significant effect on normal cells.

Due to the low level of autoimmunity in cancer patients, it is highly prone to concurrent infections, which can affect their survival periods. At this time, pathogenic bacteria that induce concurrent infections are not single, and may even contain pathogenic bacteria such as fungi, and there are few drugs in clinical practice that can achieve broad-spectrum antibacterial efficacy. Meanwhile, more and more experiments have confirmed that tumor cells are often closely related to a microbial environment, or are symbiotic with microbes, or promote growth with microbes, and thus have become one of the key cores in the development of current compound drugs to eliminate harmful microbes while killing tumor cells.

### SUMMARY

In order to solve this problem, the applicant synthesizes a compound of the present application based on a unique structure of a semi-protonated nucleotide base pair in a DNA group. Biological activities have shown that the compound has a selective anti-tumor effect that induces apoptosis. A molecular mechanism of the compound may be related to the specific inhibition of tumor cells by a transcription factor PLAGL2. In addition, the results show that the compound of the present application has broad-spectrum antimicrobial and antifungal activities, and drug-resistant bacteria, including methicillin-resistant Staphylococcus aureus, are sensitive to the compound. In an animal model of liver cancer combined with fungal infection, the compound of the present application can delay the proliferation of liver cancer cells in tumor-bearing mice, and can treat pneumonia and encephalitis caused by Cryptococcus neoformans. This study offers a new treatment option for cancer patients with complicated infections.

Provided is a protonated dimer cation, comprising or consisting of a structure of formula (A) or a resonant structure thereof, wherein “ [symbol] ” in Formula (A) is a covalent bond or a non-covalent bond,

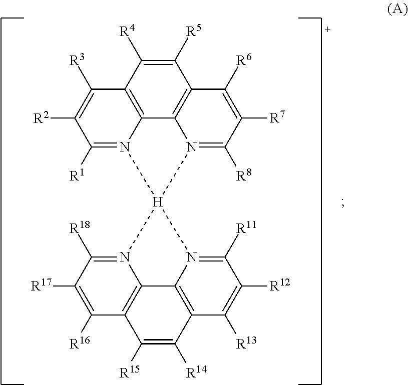

wherein, R¹, R², R³, R⁴, R⁵, R⁶, R⁷, R⁸, R¹¹, R¹², R¹³, R¹⁴, R¹⁵, R¹⁶, R¹⁷, and R¹⁸ are each independently selected from a group consisting of hydrogen, methyl, phenyl, cyano, amino, nitro, and chloro.

Provided is a salt of the present application, comprising the cation of the present application, and a first anion comprising at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion.

Provided is a method of preparing the cation of the present application or the salt of the present application, comprising: contacting a first reactant with a first acid, to obtain a first acid salt of the first reactant; contacting the first acid salt of the first reactant with a first salt comprising a first anion in an acidic environment, to obtain a first solution comprising a protonated first acid salt cation of the first reactant and the first anion; adding a second reactant to the first solution in a strong reducing and acidic environment, to obtain a second solution or a first intermediate; and adding a first base to the second solution or contacting the first base with the first intermediate, the first reactant comprising a compound of formula (C), and the second reactant comprising a compound of formula (D).

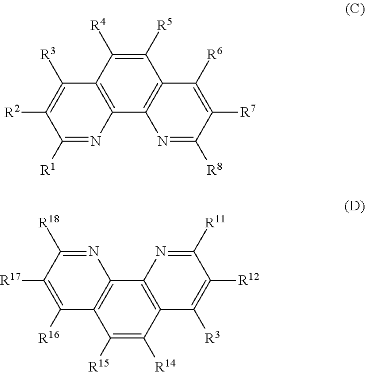

Provided is use of the protonated dimer cation of the present application or the salt of the present application in the preparation of a medicament, and in particular in the preparation of an antimicrobial agent. Provided is a method for preparing a medicament, and in particular an antimicrobial agent. The method comprises: preparing the cation of the present application or the salt of the present application.

Provided is use of the protonated dimer cation of the present application or the salt of the present application in the preparation of a medicament, and in particular in the preparation of a medicament for treating a tumor. Provided is a method for preparing a medicament, and in particular a medicament for treating a tumor. The method comprises: preparing the cation of the present application or the salt of the present application.

### BRIEF DESCRIPTION OF THE DRAWINGS

FIG. 1 a and b are respectively an ¹H nuclear magnetic resonance (NMR) spectrum and a high-performance liquid chromatography (HPLC) spectrum of a salt of Example 1.

FIG. 2 a, b, c, and d are respectively a Fourier transform infrared spectrum (FTIR), an ¹H NMR spectrum, a high-resolution mass spectrometry (HRMS) spectrum, and an HPLC spectrum of a salt of Example 4.

FIG. 3 a, b, c, and d are respectively an FTIR spectrum, an ¹H NMR spectrum, an HRMS spectrum, and an HPLC spectrum of a salt of Example 5.

FIG. 4 a, b, c, and d are respectively an FTIR spectrum, an ¹H NMR spectrum, an HRMS spectrum, and an HPLC spectrum of a salt of Example 6.

FIG. 5 a, b, c, and d are respectively an FTIR spectrum, an ¹H NMR spectrum, an HRMS spectrum, and an HPLC spectrum of a salt of Example 7.

FIG. 6 a, b, c, and d are respectively an FTIR spectrum, an ¹H NMR spectrum, an HRMS spectrum, and an HPLC spectrum of a salt of Example 8.

FIG. 7 a, b, c, and d are respectively an FTIR spectrum, an ¹H NMR spectrum, an HRMS spectrum, and an HPLC spectrum of a salt of Example 9.

FIG. 8 a, b, c, and d is ultraviolet-visible (UV-Vis) absorption spectra of aqueous solutions of salts of Examples 1, 4, 5, 6, 7, 8 and 9 at 25° C., 37° C., 60° C. and 100° C.

FIGS. 9A-9H show survival rate results of an anti-tumor test for the salt of Example 1, 5, or 8 on different tumors. FIG. 9A: human gastric adenocarcinoma cell AGS; FIG. 9B: human gastric adenocarcinoma cell HGC-27; FIG. 9C: human gastric adenocarcinoma cell MKN-45; FIG. 9D: human colon cancer cell HCT-116; FIG. 9E: human colon cancer cell SW-480; FIG. 9F: human bladder cancer cell 5637; FIG. 9G: human prostate cancer cell 22RV1; and FIG. 9H: human ovarian cancer cell Anglne. A horizontal axis represents a final concentration of the salt of Example 1, 5, or 8 in each well. A vertical axis represents a survival rate of each tumor cell.

FIGS. 10A-10C show survival rate results of an anti-tumor test for the salt of Example 1 on different tumors. FIG. 10A: human liver cancer cell SK-Hep-1, human liver cancer cell HepG2, human liver cancer cell Hep3B, human liver cancer cell SMMC-7721, human liver cancer cell LC/PRF/5, and human liver cancer cell HuH-7; FIG. 10B: human pancreatic cancer cell PANC-1, human pancreatic cancer cell BxPC3, human breast cancer cell MCF-7, human breast cancer cell SKBR3, and human breast cancer cell BT-549; and FIG. 10C: human lung cancer cell A549, human neuroblastoma cell SH, human glioma cell U251, mouse melanoma cell B16, and mouse liver cancer cell H22. Horizontal axes represent final concentration of the salt of Example 1 in each well. Vertical axes represent survival rate of each tumor cell.

FIG. 11 shows survival rate results of a cytotoxicity test for the salt of Example 1 on different normal cells (human normal hepatocytes L02, and human umbilical vein endothelial cells HUVEC).

FIG. 12 shows culture results of H22 cells and SH-SY5Y cells with the salt of Example 1.

FIG. 13 shows the results of the treatment of H22 cells with control group cells and the salt of Example 1 for 8 h, and the evaluation on the apoptosis by a flow cytometer.

FIG. 14A is diagram of a result of a disk diffusion antibacterial test in the present application, wherein “a”, “b”, “c”, “d” are respectively the salts of Examples 1, 4, 5 and 6; “cefalexin” and “streptomycin” are the respectively cefalexin or streptomycin control groups; and “control” is the negative control (DMSO). FIG. 14B shows diameters of inhibition zones of salts of various examples in the antibacterial test of the disk diffusion method in the present application.

FIG. 15A is a diagram of a result of a disk diffusion anti-mold test in the present application, wherein “a”, “b”, “c”, “d” are respectively the salts of Examples 1, 4, 5 and 6; “FLC” refers to the fluconazole control group; and “control” is the negative control (DMSO). FIG. 15B shows transmission electron microscopy (TEM) of Cryptococcus neoformans treated with a control group and the salt of Example 1, with arrows pointing to mitochondria. FIG. 15C shows a variation diagram of the cellular ATP content of Cryptococcus neoformans treated with the salt of Example 1, wherein a horizontal axis represents the time after the treatment with the salt of Example 1. FIG. 15D shows antibacterial rates of Cryptococcus neoformans treated with the salt of Example 1 of different concentrations at 8 h, 24 h, and 48 h. FIG. 15E shows TUNEL apoptosis detection results of Cryptococcus neoformans treated with the salt of Example 1 at different times, wherein red fluorescence represents apoptotic cells.

FIG. 16 is diagram of a result of a disk diffusion anti-ascomycetes test in the present application.

FIGS. 17A and 17B show tissue images (FIG. 17A) and HE staining (FIG. 17B) of mice in a mouse in-vivo toxicity test in the present application, where the mice are injected with the salt of Example 1 and a control group through tail veins.

FIG. 18 shows (A) tumor images, (B) tumor volume changes over time, (C) tumor weights, (D) mouse weights, (E) HE staining of tumors, (F) TUNEL staining of tumors, and (G) Western blot analysis of various groups in a tumor-bearing mouse test in the present application, wherein (b)-(h) of (G) are ratios of the gray values of various proteins to the gray value of β-actin, and columns in a histogram from left to right respectively represent the control group, and Example 1 low-, medium-, and high-dose treatment groups. In each group, N=6, *p<0.05, and ** p<0.01 relative to a model group.

FIG. 19 shows (A) mouse weights, (B) representative images of the brains and lungs of mice, (C) HE staining of the brains and lungs of mice, (D) culture results of tissue homogenates, (E) bacteria counts of the brains (a) and lungs (b), and (F) survival rates over time in various groups in a test for mice infected with Cryptococcus in the present application. ** p<0.01 relative to an uninfected mouse, #p<0.05, ##p<0.01 relative to a model group.

FIG. 20 shows (A) changes of tumor volume over time, (B) tumor weights, (C) tumor images, (D) representative images of the brains and lungs of mice, (E) culture results of tissue homogenates, (F) bacteria counts of brains (a) and lungs (b), and (G) survival rates over time in various groups in a test for tumor-bearing mice infected with Cryptococcus in the present application. * p<0.05, and ** p<0.01 relative to a model group.

FIG. 21 shows formula (A).

### DETAILED DESCRIPTION

As used herein, the singular term means one or more. For example, “an element” or “one element” means one or more elements. As used herein, the term “a plurality of” means at least two.

As used herein, the term “about” means approximately, in the range of about or around. When used in combination with a value range, the term “about” modifies the range by extending the limit above or below the value provided. Generally, the term “about” is used herein to give a value plus or minus 10% from the value provided. On the one hand, the term “about” means plus or minus 20% of a value of the number defined by it. For example, “about 50%” means a range of 45% to 55%. Herein, a value range defined by endpoints includes all integers and fractions within the range (for example, “1 to 5” includes 1, 1.5, 2, 2.75, 3, 3.90, 4, and 5). It should also be understood that all the integers and fractions are defined by the term “about”.

As used herein, the term “include”, “comprise” or “contain”, as a non-exclusive or open-ended term, is intended to mean that a combination (such as a device, a composition or a method) includes listed elements (such as units of the device, components of the composition, or substantive steps of the method), but other elements are not excluded. As used herein, the term “essentially consisting of . . . ” means, when used for defining a composition or a method, exclusion of other elements that have any material effect on the combination of the stated objectives, but does not exclude other elements that do not materially affect the basic and novel features of the present application. As used herein, unless otherwise described, the term “consisting of . . . ” means exclusion of the combination of other elements (units, components, substantive steps or the like), and is not intended to exclude trace amounts of unavoidable impurities. Embodiments defined by each of these connection terms fall within the scope of the present application. As a specific embodiment, a disclosed technical solution that is defined by the term “contain”, “include” or “have” shall be regarded as also disclosing a corresponding technical solution defined by the term “essentially consisting of . . . ” or “consisting of . . . ”.

As used herein the term “and/or” means and covers any or all possible combination of one or more associated listed items. When used in a list of two or more items, the term “and/or” means that any one of the listed items may be included alone or any combination of two or more of the listed items may be included. For example, if a group, combination, composition or the like is described as including (or containing) components A, B, C and/or D, the composition may include A alone; B alone; C alone; D alone; the combination of A and B; the combination of A and C; the combination of A and D; the combination of B and C; the combination of B and D; the combination of C and D; the combination of A, B, and C; the combination of A, B, and D; the combination of A, C, and D; the combination of B, C, and D; or the combination of A, B, C, and D.

As used herein, the compound or ion of the present application includes a plurality of variable groups. Those of ordinary skill in the art shall recognize that combinations of the groups that are contemplated by the present application are chemically allowed combinations of compounds or ions.

As used herein, the stereochemistry of a chiral center may be defined according to the conventions of those skilled in the art. That is, the wedged bond “ [symbol] ” is used for indicating a group in front of the plane of the paper (oriented towards the viewer), and the hashed bond . . . “ [symbol] ” is used for indicating a group behind the plane of the paper (oriented away from the viewer). It may be understood that such representations are used for indicating a specific single stereoisomer of groups represented by each chemical structure herein. Any bond that is not specifically represented by the wedged bond or the hashed bond herein shall be regarded as not specifically indicating whether the bond is in front of the plane of the paper, or behind the plane of the paper, or located in the plane of the paper. However, it does not exclude that the bond is in front of or behind the plane of the paper if chemically allowed.

As used herein, the asterisk “*” mark or numerical superscript mark (e.g., “N¹”, “N²”, “N³”, or “N⁴”) marked on an atom or a specific site in a structural formula is used only to refer to a specific atom for the purpose of description, and is not intended to refer that an atom marked with said mark has specific properties that are not described herein.

As used herein, the term “isomer” refers to compounds with the same molecular formula and different bonding nature or order of atoms or different arrangements of atoms in space. The term “stereoisomer” refers to isomers with different arrangements of atoms in space. The term “enantiomer” refers to stereoisomers with one or more asymmetric centers that are non-superimposable mirror images of each other. The term “diastereomer” refers to stereoisomers that have opposite configurations of one or more asymmetric centers and that do not belong to enantiomers. If a compound has an asymmetric center, for example, if a carbon atom is bonded to four different groups, a pair of enantiomers may exist. A configuration of an enantiomer may be characterized and designated as an R-configuration or an S-configuration by an absolute configuration of one or more asymmetric centers of the enantiomer, or the enantiomer is designated as dextrorotatory or levorotatory based on the manner in which the molecule rotates the plane of polarized light. A chiral compound may exist in the form of an enantiomer alone or a mixture of enantiomers, for example, exist in the form of a racemic mixture. The compound of the present application may contain an asymmetric center or chiral center, and exist in the form of different stereoisomers. It should be regarded that all stereoisomers of the compound of the present application include, but are not limited to, a diastereomer, an enantiomer, an atropisomer, and a mixture thereof, such as a racemic mixture, which form part of the present application.

As used herein, the term “dimer” usually refers to a compound formed by two molecules or ions, such as 1,10-phenanthroline, or a derivative or protonated ion thereof, through an interaction, especially a non-covalent interaction, for example, a compound formed by one 1,10-phenanthroline or a derivative thereof, and a protonated ion of one 1,10-phenanthroline or a derivative thereof. As used herein, the term “n dimer” usually refers to a compound formed by n molecules or ions, such as 1,10-phenanthroline, or a derivative or protonated ion thereof, through an interaction, especially a non-covalent interaction. As used herein, the term “covalent bond” refers to any bond that includes or involves electron sharing. Non-limiting examples of covalent bonds include, but are not limited to, a peptide bond, a glycosidic bond, an ester bond, and a phosphodiester bond, and in particular, includes a coordination covalent bond. As used herein, the term “non-covalent bond” includes any bond or interaction between two or more moieties that do not include or involve electron sharing. Non-limiting examples of non-covalent bonds or interactions include, but are not limited to, static electricity, T-T effects, the van der Waals force, a hydrogen bond, and the hydrophobic effect, especially the hydrogen bond. As used herein, the term “bond” includes at least a covalent bond and a non-covalent bond.

As used herein, any amino acid with chirality shall be regarded as at least referring to L-amino acid in a case that the chirality is not explicitly described. However, it does not explicitly exclude relevant embodiments of D-amino acid.

As used herein, in particular, the term “disclose or include” as described below includes, but is not limited to, the disclosure of the specification (such as, examples), drawings, or claims of the present application, and includes, but is not limited to, the scope of protection of the claims of the present application, or any one or more embodiments (such as, embodiments defined by the term “in an embodiment” or “in some embodiments” herein).

In order to further describe the technical means adopted by the present application to achieve the intended objective and functions, the following describes specific embodiments, structures, features, and functions of the present application in detail with reference to the accompanying drawings and preferred examples.

### Protonated Cation of the Present Application

In a first aspect, the present application provides a protonated dimer cation, comprising a structure of formula (A) or a resonant structure thereof, or consisting of the structure of formula (A) or the resonant structure thereof, wherein “ [symbol] ” in Formula (A) shall be deemed to comprise a chemically allowed force, comprising but not limited to a covalent bond or a non-covalent bond, that enables the protonated dimer cation of the present application to exist stably.

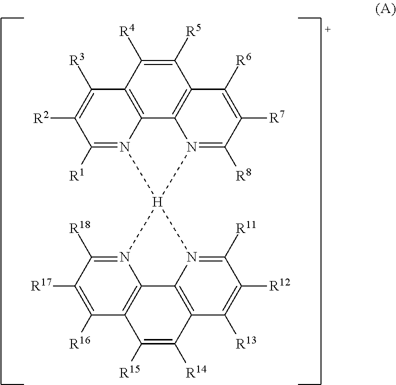

In Formula (A), it can be regarded, if chemically allowed, as a resonant structure in which H* has a first bond with one of N¹ and N² and a second bond with one of N³ and N⁴, as shown in the structure of (A1) to a structure of (A8). In particular, one of the first bond and the second bond may be a covalent bond and the other is a hydrogen bond. For example, the structure of formula (A) further comprise a structure of (A1) with a covalent bond between H* and N¹ and a hydrogen bond between H* and N³; a structure of (A2) with a covalent bond between H* and N¹ and a hydrogen bond between H* and N⁴; a structure of (A3) with a covalent bond between H* and N² and a hydrogen bond between H* and N³; a structure of (A4) with a covalent bond between H* and N² and a hydrogen bond between H* and N⁴; a structure of (A5) with a covalent bond between H* and N³ and a hydrogen bond between H* and N¹; a structure of (A6) with a covalent bond between H* and N³ and a hydrogen bond between H* and N²; a structure of (A7) with a covalent bond between H* and N⁴ and a hydrogen bond between H* and N¹; and a structure of (A8) with a covalent bond between H* and N⁴ and a hydrogen bond between H* and N². In the structure of formula (A1) to the structure of formula (A8), H* may also have some degree of force with two other nitrogen atoms (N² and N⁴ in the case of formula (A1)) whose bonds are not drawn, as long as they are chemically reasonable.

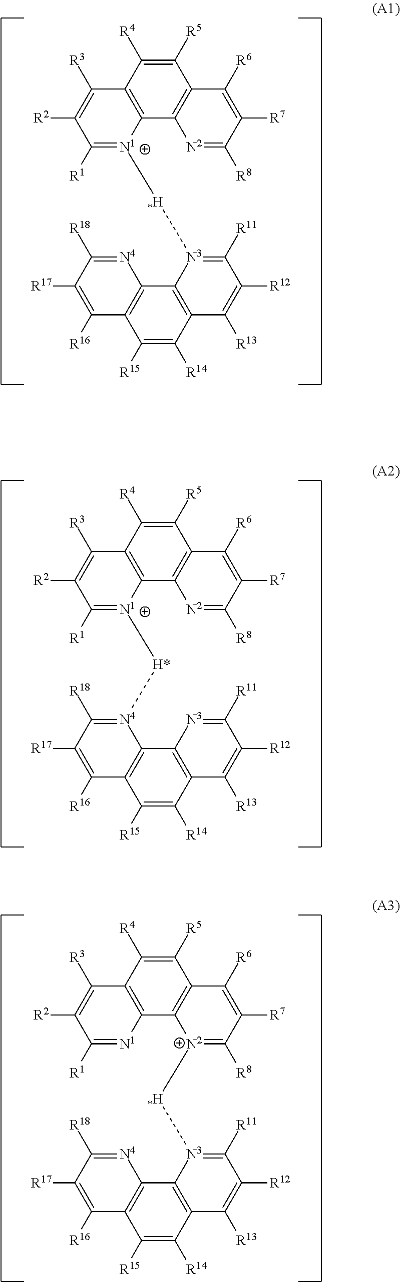

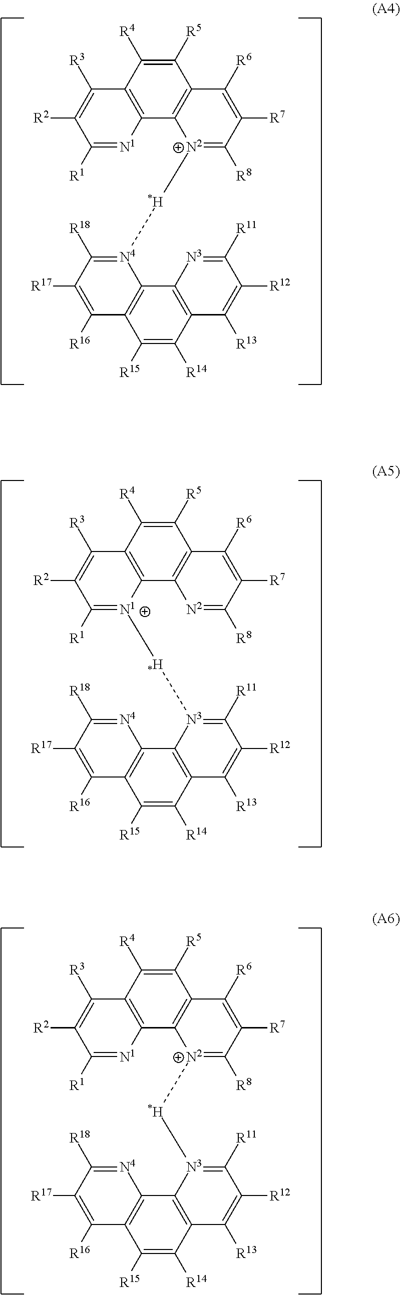

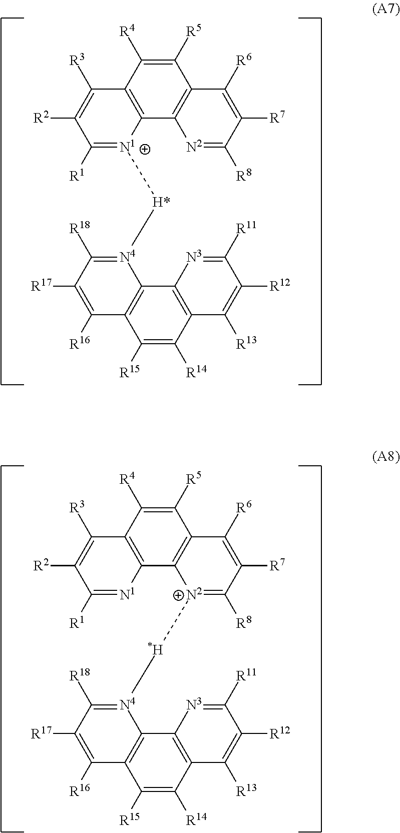

Although the structure of (A) is drawn on a plane, those skilled in the art should understand that the structure of (A) is not necessarily a planar structure, and two 1,10-phenanthroline (or a derivative thereof) moieties in this structure may be formed at any dihedral angle, as long as the resulting structure is chemically reasonable.

In the present application, one or more of the structures of formula (A1) to formula (A8) in any disclosure or scope, which include any particular R¹, R², R³, R⁴, R⁵, R⁶, R⁷, R⁸, R¹¹, R¹², R¹³, R¹⁴, R¹⁵, R¹⁶, R¹⁷, and/or R¹⁸, shall be deemed to disclose or include other structures among the structures represented by the corresponding formula (A1) to formula (A8), and to disclose or include the corresponding structure of formula (A).

In some embodiments, R¹, R², R³, R⁴, R⁵, R⁶, R⁷, R⁸, R¹¹, R¹², R¹³, R¹⁴, R¹⁵, R¹⁶, R¹⁷, and R¹⁸ are each independently selected from a group consisting of hydrogen, methyl, phenyl, cyano, amino, nitro, and chloro.

In some embodiments, R¹ is selected from a group consisting of hydrogen, methyl, phenyl, cyano, amino, nitro, and chloro. In some embodiments, R¹ is selected from a group consisting of hydrogen, methyl, phenyl, cyano, amino, and chloro. In some embodiments, R¹ is selected from a group consisting of hydrogen, methyl, cyano, and chloro. In some embodiments, R¹ is selected from a group consisting of hydrogen and methyl. In some embodiments, R¹ is hydrogen. In some embodiments, R¹ is methyl. In some embodiments, R¹ is phenyl. In some embodiments, R¹ is cyano. In some embodiments, R¹ is amino. In some embodiments, R¹ is nitro. In some embodiments, R¹ is chloro.

In some embodiments, R² is selected from a group consisting of hydrogen, methyl, phenyl, cyano, amino, nitro, and chloro. In some embodiments, R² is selected from a group consisting of hydrogen, methyl, phenyl, amino, and chloro. In some embodiments, R² is selected from a group consisting of hydrogen and methyl. In some embodiments, R² is hydrogen. In some embodiments, R² is methyl. In some embodiments, R² is phenyl. In some embodiments, R² is cyano. In some embodiments, R² is amino. In some embodiments, R² is nitro. In some embodiments, R² is chloro.

In some embodiments, R³ is selected from a group consisting of hydrogen, methyl, phenyl, cyano, amino, nitro, and chloro. In some embodiments, R³ is selected from a group consisting of hydrogen, methyl, phenyl, amino, and chloro. In some embodiments, R³ is selected from a group consisting of hydrogen, methyl, and phenyl. In some embodiments, R³ is selected from a group consisting of hydrogen and methyl. In some embodiments, R³ is hydrogen. In some embodiments, R³ is methyl. In some embodiments, R³ is phenyl. In some embodiments, R³ is cyano. In some embodiments, R³ is amino. In some embodiments, R³ is nitro. In some embodiments, R³ is chloro.

In some embodiments, R⁴ is selected from a group consisting of hydrogen, methyl, phenyl, cyano, amino, nitro, and chloro. In some embodiments, R⁴ is selected from a group consisting of hydrogen, methyl, phenyl, amino, nitro, and chloro. In some embodiments, R⁴ is selected from a group consisting of hydrogen, amino, and nitro. In some embodiments, R⁴ is hydrogen. In some embodiments, R⁴ is methyl. In some embodiments, R⁴ is phenyl. In some embodiments, R⁴ is cyano. In some embodiments, R⁴ is amino. In some embodiments, R⁴ is nitro. In some embodiments, R⁴ is chloro.

In some embodiments, R⁵ is selected from a group consisting of hydrogen, methyl, phenyl, cyano, amino, nitro, and chloro. In some embodiments, R⁵ is selected from a group consisting of hydrogen, methyl, phenyl, amino, nitro, and chloro. In some embodiments, R⁵ is selected from a group consisting of hydrogen, amino, and nitro. In some embodiments, R⁵ is hydrogen. In some embodiments, R⁵ is methyl. In some embodiments, R⁵ is phenyl. In some embodiments, R⁵ is cyano. In some embodiments, R⁵ is amino. In some embodiments, R⁵ is nitro. In some embodiments, R⁵ is chloro.

In some embodiments, R⁶ is selected from a group consisting of hydrogen, methyl, phenyl, cyano, amino, nitro, and chloro. In some embodiments, R⁶ is selected from a group consisting of hydrogen, methyl, phenyl, amino, and chloro. In some embodiments, R⁶ is selected from a group consisting of hydrogen, methyl, and phenyl. In some embodiments, R⁶ is selected from a group consisting of hydrogen and methyl. In some embodiments, R⁶ is hydrogen. In some embodiments, R⁶ is methyl. In some embodiments, R⁶ is phenyl. In some embodiments, R⁶ is cyano. In some embodiments, R⁶ is amino. In some embodiments, R⁶ is nitro. In some embodiments, R⁶ is chloro.

In some embodiments, R⁷ is selected from a group consisting of hydrogen, methyl, phenyl, cyano, amino, nitro, and chloro. In some embodiments, R⁷ is selected from a group consisting of hydrogen, methyl, phenyl, amino, and chloro. In some embodiments, R⁷ is selected from a group consisting of hydrogen and methyl. In some embodiments, R⁷ is hydrogen. In some embodiments, R⁷ is methyl. In some embodiments, R⁷ is phenyl. In some embodiments, R⁷ is cyano. In some embodiments, R⁷ is amino. In some embodiments, R⁷ is nitro. In some embodiments, R⁷ is chloro.

In some embodiments, R⁸ is selected from a group consisting of hydrogen, methyl, phenyl, cyano, amino, nitro, and chloro. In some embodiments, R⁸ is selected from a group consisting of hydrogen, methyl, phenyl, cyano, amino, and chloro. In some embodiments, R⁸ is selected from a group consisting of hydrogen, methyl, cyano, and chloro. In some embodiments, R⁸ is selected from a group consisting of hydrogen and methyl. In some embodiments, R⁸ is hydrogen. In some embodiments, R⁸ is methyl. In some embodiments, R⁸ is phenyl. In some embodiments, R⁸ is cyano. In some embodiments, R⁸ is amino. In some embodiments, R⁸ is nitro. In some embodiments, R⁸ is chloro.

In some embodiments, R¹¹ is selected from a group consisting of hydrogen, methyl, phenyl, cyano, amino, nitro, and chloro. In some embodiments, R¹¹ is selected from a group consisting of hydrogen, methyl, phenyl, cyano, amino, and chloro. In some embodiments, R¹¹ is selected from a group consisting of hydrogen, methyl, cyano, and chloro. In some embodiments, R¹¹ is selected from a group consisting of hydrogen and methyl. In some embodiments, R¹¹ is hydrogen. In some embodiments, R¹¹ is methyl. In some embodiments, R¹¹ is phenyl. In some embodiments, R¹¹ is cyano. In some embodiments, R¹¹ is amino. In some embodiments, R¹¹ is nitro. In some embodiments, R¹¹ is chloro.

In some embodiments, R¹² is selected from a group consisting of hydrogen, methyl, phenyl, cyano, amino, nitro, and chloro. In some embodiments, R¹² is selected from a group consisting of hydrogen, methyl, phenyl, amino, and chloro. In some embodiments, R¹² is selected from a group consisting of hydrogen and methyl. In some embodiments, R¹² is hydrogen. In some embodiments, R¹² is methyl. In some embodiments, R¹² is phenyl. In some embodiments, R¹² is cyano. In some embodiments, R¹² is amino. In some embodiments, R¹² is nitro. In some embodiments, R¹² is chloro.

In some embodiments, R¹³ is selected from a group consisting of hydrogen, methyl, phenyl, cyano, amino, nitro, and chloro. In some embodiments, R¹³ is selected from a group consisting of hydrogen, methyl, phenyl, amino, and chloro. In some embodiments, R¹³ is selected from a group consisting of hydrogen, methyl, and phenyl. In some embodiments, R¹³ is selected from a group consisting of hydrogen and methyl. In some embodiments, R¹³ is hydrogen. In some embodiments, R¹³ is methyl. In some embodiments, R¹³ is phenyl. In some embodiments, R¹³ is cyano. In some embodiments, R¹³ is amino. In some embodiments, R¹³ is nitro. In some embodiments, R¹³ is chloro.

In some embodiments, R¹⁴ is selected from a group consisting of hydrogen, methyl, phenyl, cyano, amino, nitro, and chloro. In some embodiments, R¹⁴ is selected from a group consisting of hydrogen, methyl, phenyl, amino, nitro, and chloro. In some embodiments, R¹⁴ is selected from a group consisting of hydrogen, amino, and nitro. In some embodiments, R¹⁴ is hydrogen. In some embodiments, R¹⁴ is methyl. In some embodiments, R¹⁴ is phenyl. In some embodiments, R¹⁴ is cyano. In some embodiments, R¹⁴ is amino. In some embodiments, R¹⁴ is nitro. In some embodiments, R¹⁴ is chloro.

In some embodiments, R¹⁵ is selected from a group consisting of hydrogen, methyl, phenyl, cyano, amino, nitro, and chloro. In some embodiments, R¹⁵ is selected from a group consisting of hydrogen, methyl, phenyl, amino, nitro, and chloro. In some embodiments, R¹⁵ is selected from a group consisting of hydrogen, amino, and nitro. In some embodiments, R¹⁵ is hydrogen. In some embodiments, R¹⁵ is methyl. In some embodiments, R¹⁵ is phenyl. In some embodiments, R¹⁵ is cyano. In some embodiments, R¹⁵ is amino. In some embodiments, R¹⁵ is nitro. In some embodiments, R¹⁵ is chloro.

In some embodiments, R¹⁶ is selected from a group consisting of hydrogen, methyl, phenyl, cyano, amino, nitro, and chloro. In some embodiments, R¹⁶ is selected from a group consisting of hydrogen, methyl, phenyl, amino, and chloro. In some embodiments, R¹⁶ is selected from a group consisting of hydrogen, methyl, and phenyl. In some embodiments, R¹⁶ is selected from a group consisting of hydrogen and methyl. In some embodiments, R¹⁶ is hydrogen. In some embodiments, R¹⁶ is methyl. In some embodiments, R¹⁶ is phenyl. In some embodiments, R¹⁶ is cyano. In some embodiments, R¹⁶ is amino. In some embodiments, R¹⁶ is nitro. In some embodiments, R¹⁶ is chloro.

In some embodiments, R¹⁷ is selected from a group consisting of hydrogen, methyl, phenyl, cyano, amino, nitro, and chloro. In some embodiments, R¹⁷ is selected from a group consisting of hydrogen, methyl, phenyl, amino, and chloro. In some embodiments, R¹⁷ is selected from a group consisting of hydrogen and methyl. In some embodiments, R¹⁷ is hydrogen. In some embodiments, R¹⁷ is methyl. In some embodiments, R¹⁷ is phenyl. In some embodiments, R¹⁷ is cyano. In some embodiments, R¹⁷ is amino. In some embodiments, R¹⁷ is nitro. In some embodiments, R¹⁷ is chloro.

In some embodiments, R¹⁸ is selected from a group consisting of hydrogen, methyl, phenyl, cyano, amino, nitro, and chloro. In some embodiments, R¹⁸ is selected from a group consisting of hydrogen, methyl, phenyl, cyano, amino, and chloro. In some embodiments, R¹⁸ is selected from a group consisting of hydrogen, methyl, cyano, and chloro. In some embodiments, R¹⁸ is selected from a group consisting of hydrogen and methyl. In some embodiments, R¹⁸ is hydrogen. In some embodiments, R¹⁸ is methyl. In some embodiments, R¹⁸ is phenyl. In some embodiments, R¹⁸ is cyano. In some embodiments, R¹⁸ is amino. In some embodiments, R¹⁸ is nitro. In some embodiments, R¹⁸ is chloro.

In some embodiments, R¹ and R⁸ are the same. In some embodiments, R² and R⁷ are the same. In some embodiments, R³ and R⁶ are the same. In some embodiments, R⁴ and R⁵ are the same. In some embodiments, R¹¹ and R¹⁸ are the same. In some embodiments, R¹² and R¹⁷ are the same. In some embodiments, R¹³ and R¹⁶ are the same. In some embodiments, R¹⁴ and R¹⁵ are the same. In some embodiments, R¹ and R¹¹ are the same. In some embodiments, R² and R¹² are the same. In some embodiments, R³ and R¹³ are the same. In some embodiments, R⁴ and R¹⁴ are the same. In some embodiments, R⁵ and R¹⁵ are the same. In some embodiments, R⁶ and R¹⁶ are the same. In some embodiments, R⁷ and R¹⁷ are the same. In some embodiments, R⁸ and R¹⁸ are the same. In some embodiments, R¹ and R¹⁸ are the same. In some embodiments, R² and R¹⁷ are the same. In some embodiments, R³ and R¹⁶ are the same. In some embodiments, R⁴ and R¹⁵ are the same. In some embodiments, R⁵ and R¹⁴ are the same. In some embodiments, R⁶ and R¹³ are the same. In some embodiments, R⁷ and R¹² are the same. In some embodiments, R⁸ and R¹¹ are the same. In some embodiments, R¹, R⁸, R¹¹ and R¹⁸ are the same. In some embodiments, R², R⁷, R¹² and R¹⁷ are the same. In some embodiments, R³, R⁶, R¹³ and R¹⁶ are the same. In some embodiments, R⁴, R⁵, R¹⁴ and R¹⁵ are the same.

In some embodiments, one of R¹ and R⁸ is hydrogen, and the other is selected from a group consisting of methyl, phenyl, cyano, amino, nitro, and chloro. In some embodiments, one of R¹ and R⁸ is hydrogen, and the other is selected from a group consisting of methyl, phenyl, cyano, amino, and chloro. In some embodiments, one of R¹ and R⁸ is hydrogen, and the other is selected from a group consisting of cyano and chloro. In some embodiments, one of R¹¹ and R¹⁸ is hydrogen, and the other is selected from a group consisting of methyl, phenyl, cyano, amino, nitro, and chloro. In some embodiments, one of R¹¹ and R¹⁸ is hydrogen, and the other is selected from a group consisting of methyl, phenyl, cyano, amino, and chloro. In some embodiments, one of R¹¹ and R¹⁸ is hydrogen, and the other is selected from a group consisting of cyano and chloro. In some embodiments, one of R⁴ and R⁵ is hydrogen, and the other is selected from a group consisting of methyl, phenyl, cyano, amino, nitro, and chloro. In some embodiments, one of R⁴ and R⁵ is hydrogen, and the other is selected from a group consisting of methyl, phenyl, amino, nitro, and chloro. In some embodiments, one of R⁴ and R⁵ is hydrogen, and the other is selected from a group consisting of amino and nitro. In some embodiments, one of R¹⁴ and R¹⁵ is hydrogen, and the other is selected from a group consisting of methyl, phenyl, cyano, amino, nitro, and chloro. In some embodiments, one of R¹⁴ and R¹⁵ is hydrogen, and the other is selected from a group consisting of methyl, phenyl, amino, nitro, and chloro. In some embodiments, one of R¹⁴ and R¹⁵ is hydrogen, and the other is selected from a group consisting of amino and nitro.

In some embodiments, R¹ is hydrogen, R² is hydrogen, R³ is hydrogen, and R⁴ is hydrogen. In some embodiments, R⁵ is hydrogen, R⁶ is hydrogen, R⁷ is hydrogen, and R⁸ is hydrogen. In some embodiments, R¹¹ is hydrogen, R¹² is hydrogen, R¹³ is hydrogen, and R¹⁴ is hydrogen. In some embodiments, R¹⁵ is hydrogen, R¹⁶ is hydrogen, R¹⁷ is hydrogen, and R¹⁸ is hydrogen. In some embodiments, R¹ is methyl, R² is hydrogen, R³ is hydrogen, and R⁴ is hydrogen. In some embodiments, R⁵ is hydrogen, R⁶ is hydrogen, R⁷ is hydrogen, and R⁸ is methyl. In some embodiments, R¹¹ is methyl, R¹² is hydrogen, R¹³ is hydrogen, and R¹⁴ is hydrogen. In some embodiments, R¹⁵ is hydrogen, R¹⁶ is hydrogen, R¹⁷ is hydrogen, and R¹⁸ is methyl. In some embodiments, R¹ is hydrogen, R² is methyl, R³ is methyl, and R⁴ is hydrogen. In some embodiments, R⁵ is hydrogen, R⁶ is methyl, R⁷ is methyl, and R⁸ is hydrogen. In some embodiments, R¹¹ is hydrogen, R¹² is methyl, R¹³ is methyl, and R¹⁴ is hydrogen. In some embodiments, R¹⁵ is hydrogen, R¹⁶ is methyl, R¹⁷ is methyl, and R¹⁸ is hydrogen. In some embodiments, R¹ is hydrogen, R² is hydrogen, R³ is phenyl, and R⁴ is hydrogen. In some embodiments, R⁵ is hydrogen, R⁶ is phenyl, R⁷ is hydrogen, and R⁸ is hydrogen. In some embodiments, R¹¹ is hydrogen, R¹² is hydrogen, R¹³ is phenyl, and R¹⁴ is hydrogen. In some embodiments, R¹⁵ is hydrogen, R¹⁶ is phenyl, R¹⁷ is hydrogen, and R¹⁸ is hydrogen. In some embodiments, R¹ is hydrogen, R² is hydrogen, R³ is hydrogen, and R⁴ is amino. In some embodiments, R⁵ is amino, R⁶ is hydrogen, R⁷ is hydrogen, and R⁸ is hydrogen. In some embodiments, R¹¹ is hydrogen, R¹² is hydrogen, R¹³ is hydrogen, and R¹⁴ is amino. In some embodiments, R¹⁵ is amino, R¹⁶ is hydrogen, R¹⁷ is hydrogen, and R¹⁸ is hydrogen. In some embodiments, R¹ is hydrogen, R² is hydrogen, R³ is hydrogen, and R⁴ is nitro. In some embodiments, R⁵ is nitro, R⁶ is hydrogen, R⁷ is hydrogen, and R⁸ is hydrogen. In some embodiments, R¹¹ is hydrogen, R¹² is hydrogen, R¹³ is hydrogen, and R¹⁴ is nitro. In some embodiments, R¹⁵ is nitro, R¹⁶ is hydrogen, R¹⁷ is hydrogen, and R¹⁸ is hydrogen. In some embodiments, R¹ is chloro, R² is hydrogen, R³ is hydrogen, and R⁴ is hydrogen. In some embodiments, R⁵ is hydrogen, R⁶ is hydrogen, R⁷ is hydrogen, and R⁸ is chloro. In some embodiments, R¹¹ is chloro, R¹² is hydrogen, R¹³ is hydrogen, and R¹⁴ is hydrogen. In some embodiments, R¹⁵ is hydrogen, R¹⁶ is hydrogen, R¹⁷ is hydrogen, and R¹⁸ is chloro. In some embodiments, R¹ is cyano, R² is hydrogen, R³ is hydrogen, and R⁴ is hydrogen. In some embodiments, R⁵ is hydrogen, R⁶ is hydrogen, R⁷ is hydrogen, and R⁸ is cyano. In some embodiments, R¹¹ is cyano, R¹² is hydrogen, R¹³ is hydrogen, and R¹⁴ is hydrogen. In some embodiments, R¹⁵ is hydrogen, R¹⁶ is hydrogen, R¹⁷ is hydrogen, and R¹⁸ is cyano.

In some embodiments, R¹ and R¹¹ are each independently selected from a group consisting of hydrogen, methyl, cyano, and chloro; R², R⁷, R¹², and R¹⁷ are each independently selected from a group consisting of hydrogen and methyl; R³, R⁶, R¹³, and R¹⁶ are each independently selected from a group consisting of hydrogen, methyl, and phenyl; R⁴ and R¹⁴ are each independently selected from a group consisting of hydrogen, nitro, and amino; R⁵ and R¹⁵ are hydrogen; and R⁸ and R¹⁸ are each independently selected from a group consisting of hydrogen and methyl. In some embodiments, R¹ and R¹¹ are the same; R², R⁷, R¹², and R¹⁷ are the same; R³, R⁶, R¹³, and R¹⁶ are the same; R⁴ and R¹⁴ are the same; R⁵ and R¹⁵ are the same; and R⁸ and R¹⁸ are the same. In some embodiments, R¹, R⁸, R¹¹, and R¹⁸ are hydrogen; R², R³, R⁶, R⁷, R¹², R¹³, R¹⁶, and R¹⁷ are hydrogen; and/or, R⁴, R⁵, R¹⁴, and R¹⁵ are hydrogen.

Any chemical structure that is disclosed or contained in the present application and specified by any particular R¹, R², R³, R⁴, R⁵, R⁶, R⁷, R⁸, R¹¹, R¹², R¹³, R¹⁴, R¹⁵, R¹⁶, R¹⁷, and/or R¹⁸ selected from formula (A) and/or other structural formulas shall be regarded as further disclosing and containing a stereoisomer of the specified chemical structure. Any chemical structure disclosed or contained in the present application, especially in examples, shall be regarded as further disclosing or containing a stereoisomer of the chemical structure.

In the present application, if the stereochemistry of any one or more chiral centers is not specified, it shall be regarded as disclosing or containing possible stereoisomers of the chiral center or combinations or mixtures of the stereoisomers. For example, if the stereochemistry of a chiral center is not specified, it shall be regarded as respectively disclosing or containing a chemical structure of a chiral center in an (R)-configuration, a chemical structure of a chiral center in an(S)-configuration, and a combination of the chiral center in any ratio, including a racemic mixture. For example, if the stereochemistry of two chiral centers is not specified, it shall be regarded as respectively disclosing or containing a chemical structure of the two chiral centers in an (R,R)-configuration, a chemical structure of the two chiral centers in an (S,S)-configuration, a chemical structure of the two chiral centers in an (R,S)-configuration, a chemical structure of the two chiral centers in an (S,R)-configuration, and a combination of the two chiral centers in any ratio.

Any chemical structure that is disclosed and contained in the present application and specified by any particular R¹, R², R³, R⁴, R⁵, R⁶, R⁷, R⁸, R¹¹, R¹², R¹³, R¹⁴, R¹⁵, R¹⁶, R¹⁷, and/or R¹⁸ selected from formula (A) and/or other structural formulas shall be regarded as further disclosing and containing a chemically allowed tautomer of the specified chemical structure. Any chemical structure disclosed or contained in the present application, especially in examples, shall be regarded as further disclosing or containing a chemically allowed stereoisomer of the chemical structure.

### Salt of the Present Application

In an aspect, the present application provides a salt.

In some embodiments, the salt of the present application comprises a protonated dimer cation of the present application, which especially comprises the protonated dimer cation of the present application and a first anion. For example, the salt of the present application may be of formula (B), wherein X is the first anion.

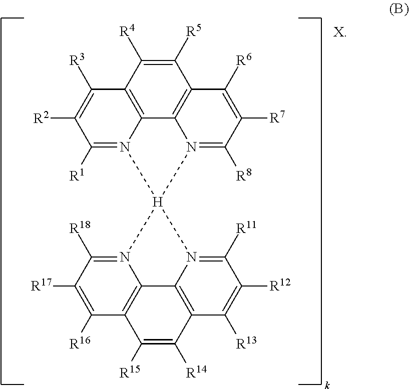

Through the first anion of the present application, the cation of the present application is enabled to exist stably in the form of a salt.

In some embodiments, the first anion (X in formula (B)) comprises or consists of at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion. In some embodiments, the first anion comprises or consists of at least one selected from a group consisting of a hexafluorophosphate ion, a hexafluorosilicate ion, a chloride ion, and a sulfate ion. In some embodiments, the first anion is a fluoride ion. In some embodiments, the first anion is a chloride ion. In some embodiments, the first anion is a bromide ion. In some embodiments, the first anion is an iodide ion. In some embodiments, the first anion is a sulfide ion. In some embodiments, the first anion is a nitrate ion. In some embodiments, the first anion is a sulfate ion. In some embodiments, the first anion is a sulfite ion. In some embodiments, the first anion is a thiosulfate ion. In some embodiments, the first anion is a persulfate ion. In some embodiments, the first anion is a selenate ion. In some embodiments, the first anion is a phosphate ion. In some embodiments, the first anion is a carbonate ion. In some embodiments, the first anion is a hexafluorophosphate ion. In some embodiments, the first anion is a hexafluorosilicate ion. In some embodiments, the first anion is an acetate ion. In some embodiments, the first anion is a sulfonate ion such as a sulfamate ion. In some embodiments, the first anion is a benzoate ion. In some embodiments, the first anion is a polyphosphate ion.

A value of a parameter k is according to the valence of the protonated dimer cation of the present application and the valence of the first anion. In a case of the protonated dimer cation of the present application with a monovalent positive electron, the value of the parameter k is equal to the valence of the first anion. In some embodiments, k is 1. In some embodiments, k is 2. In some embodiments, k is 3.

### Method of Preparing the Protonated Dimer Cation of the Present Application or Salt of the Present Application

In some embodiments, a method of preparing of a salt of the protonated dimer cation of the present application, that is, the salt of the present application, may comprise the following steps.

### Salt Formation of a First Reactant

A first reactant is contacted with a first acid. Then, a first acid salt of the first reactant is obtained. In some embodiments, the first acid salt of the first reactant comprises the first reactant and the first acid, and/or a conjugate base (a conjugate base of the first acid) of a protonated first reactant and the first acid, or consists of the first reactant and the first acid, and/or the conjugate base.

In some embodiments, the first reactant comprises a compound of formula (C) or basically consists of the compound, and particularly, consists of the compound. In some embodiments, the first acid salt of the first reactant comprises a salt of formula (C1) or basically consists of the salt, and particularly, consists of the salt. A is a conjugate base of the first acid, a is the valence of the conjugate base of the first acid, and p is the number of equivalents of HaA. R¹, R², R³, R⁴, R⁵, R⁶, R⁷, and R⁸ in formula (C) or formula (C1) may coincide with corresponding moieties or parameters of a desired cation (formula (A)) or salt (formula (B)) according to examples of the present application.

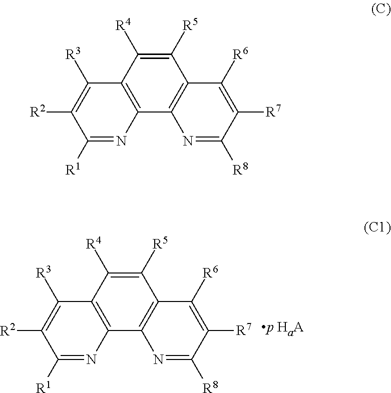

In some embodiments, the first reactant is contacted with the first acid in the presence of a first solvent, and particularly, in the first solvent. In some embodiments, the first acid is at least one selected from a group consisting of nitric acid, phosphoric acid, sulfuric acid, hydrofluoric acid, hydrochloric acid, hydrobromic acid, and hydroiodic acid, especially hydrochloric acid. That is, the conjugate base (A in formula (C1)) of the first acid is at least one selected from a group consisting of a nitrate ion, a phosphate ion, a hydrogen phosphate ion, a dihydrogen phosphate ion, a sulfate ion, a hydrogen sulfate ion, a fluoride ion, a chloride ion, a bromide ion, and an iodide ion, especially a chloride ion. In some embodiments, the first solvent is water. In formula (C1), the parameters p and a are according to the type of the first acid. For example, in some embodiments, the first acid is hydrochloric acid, and p is 1.

Unless otherwise further defined, the term “conjugate acid” herein is intended to include a Brønsted-Lowry conjugate acid of a compound to which the term is directed. Furthermore, it does not only include a first conjugate acid obtained in a case that the compound acquires a proton, but also include, if chemically allowed, an acid obtained in a case that the compound further acquires more protons, such as a second conjugate acid obtained in a case that the first conjugate further acquires a proton, and a third conjugate acid obtained in a case that the second conjugate acid much further acquires a proton, and so on. Unless otherwise further defined, the term “conjugate base” herein is intended to include a Brønsted-Lowry conjugate base of a compound to which the term is directed. Further, it does not only include a first conjugate base obtained in a case that the compound loses a proton, but also includes, if chemically allowed, a base obtained in a case that the compound further loses more protons, such as a second conjugate base obtained in a case that the first conjugate base further loses a proton, and a third conjugate base obtained in a case that the second conjugate base much further loses a proton, and so on. For example, unless otherwise further defined, a conjugate base of sulfuric acid herein shall be regarded as including not only a hydrogen sulfate ion, but also a sulfate ion. For example, unless otherwise further defined, a conjugate base of phosphoric acid herein shall be regarded as including not only a hydrogen phosphate ion, but also a dihydrogen phosphate ion and a phosphate ion.

In some embodiments, the first acid is in excess relative to the first reactant. In some embodiments, a molar ratio of the first reactant to the first acid is 1:2 to 1:10, particularly 1:2.5 to 1:8, more particularly 1:4 to 1:6, and more particularly about 1:5.

In some embodiments, a concentration of the first acid in the first solvent is more than 1 M. In some embodiments, the concentration of the first acid in the first solvent is 1 M to 5 M. In some embodiments, the concentration of the first acid in the first solvent is about 2 M.

In some embodiments, the first reactant is contacted with the first acid at a first temperature. In some embodiments, the first temperature is 0° C. to 70° C. In some embodiments, the first temperature is 10° C. to 40° C., more particularly 20° C. to 30° C., and more particularly about 25° C. In some embodiments, the first temperature is 20° C. to 50° C., more particularly 30° C. to 40° C., and more particularly about 37° C. In some embodiments, the first temperature is 0° C. to 20° C., more particularly 0° C. to 10° C., and more particularly about 4° C. In some embodiments, the first temperature is 40° C. to 70° C., more particularly 50° C. to 60° C., and more particularly about 60° C.

In some embodiments, after the first reactant is contacted with the first acid, heating and recrystallization are performed to obtain the first acid salt of the first reactant. In some embodiments, the heating temperature is 70° C. to 100° C., particularly 80° C. to 90° C., and more particularly about 85° C.

### Protonation of the First Acid Salt of the First Reactant

The first acid salt of the first reactant is contacted with a first salt that comprises a first anion, and particularly, consists of a first cation and the first anion in a first acidic environment. Then, a first solution that comprises a protonated first acid salt cation of the first reactant and the first anion is obtained, as shown in formula (C2).

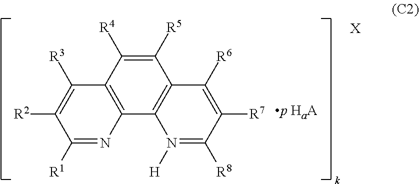

X and k in formula (C2) may coincide with corresponding moieties or parameters of a desired salt (formula (B)) according to an example of the present application.

In some embodiments, the first salt comprises or consists of at least one selected from a group consisting of ammonium fluoride, ammonium chloride, ammonium bromide, ammonium iodide, ammonium sulfide, ammonium nitrate, ammonium sulfate, ammonium sulfite, ammonium thiosulfate, ammonium persulfate, ammonium selenate, ammonium phosphate, ammonium carbonate, ammonium hexafluorophosphate, ammonium hexafluorosilicate, ammonium acetate, any ammonium sulfonate, ammonium benzoate, and ammonium polyphosphate. In some embodiments, the first salt comprises or consists of at least one selected from a group consisting of ammonium hexafluorophosphate, ammonium hexafluorosilicate, ammonium chloride, and ammonium sulfate. In some embodiments, the first salt is ammonium fluoride. In some embodiments, the first salt is ammonium chloride. In some embodiments, the first salt is ammonium bromide. In some embodiments, the first salt is ammonium iodide. In some embodiments, the first salt is ammonium sulfide. In some embodiments, the first salt is ammonium nitrate. In some embodiments, the first salt is ammonium sulfate. In some embodiments, the first salt is ammonium sulfite. In some embodiments, the first salt is ammonium thiosulfate. In some embodiments, the first salt is ammonium persulfate. In some embodiments, the first salt is ammonium selenate. In some embodiments, the first salt is ammonium phosphate. In some embodiments, the first salt is ammonium carbonate. In some embodiments, the first salt is ammonium hexafluorophosphate. In some embodiments, the first salt is ammonium hexafluorosilicate. In some embodiments, the first salt is ammonium acetate. In some embodiments, the first salt is any ammonium sulfonate such as ammonium sulfamate. In some embodiments, the first salt is ammonium benzoate. In some embodiments, the first salt is ammonium polyphosphate.

In some embodiments, the first acid salt of the first reactant is contacted with the first salt in the presence of a second solvent, and particularly, in the second solvent. In some embodiments, the second solvent is water. In some embodiments, the first cation is an ammonium ion. Here, the ammonium ion, as the first cation, may be taken as a hydrogen donor.

In some embodiments, the first salt is in excess relative to the first acid salt of the first reactant. In some embodiments, a molar ratio of the first acid salt of the first reactant to the first salt is 1:2 to 1:10, particularly 1:2.5 to 1:8, more particularly 1:4 to 1:6, and more particularly about 1:5.

In some embodiments, the first solution further comprises a conjugate base of the first acid. In some embodiments, the first solution further comprises the first cation. In some embodiments, the first solution further comprises the second solvent. In some embodiments, the first solution essentially, and particularly, consists of the protonated first acid salt cation of the first reactant, the first cation, the first anion, the conjugate base of the first acid, and the second solvent.

In some embodiments, the first acid salt of the first reactant is contacted with the first salt at a second temperature. In some embodiments, the second temperature is 0° C. to 70° C. In some embodiments, the second temperature is 10° C. to 40° C., more particularly 20° C. to 30° C., and more particularly about 25° C. In some embodiments, the second temperature is 20° C. to 50° C., more particularly 30° C. to 40° C., and more particularly about 37° C. In some embodiments, the second temperature is 0° C. to 20° C., more particularly 0° C. to 10° C., and more particularly about 4° C. In some embodiments, the second temperature is 40° C. to 70° C., more particularly 50° C. to 60° C., and more particularly about 60° C. In some embodiments, the first temperature and the second temperature are the same.

In some embodiments, the pH of the first acidic environment is less than or equal to 6. In some embodiments, the pH of the first acidic environment is 4 to 6, particularly 4.5 to 5.5, particularly 4.8 to 5.2, particularly 4.9 to 5.1, and more particularly about 5. Generally, for the first anion that is less acidic or more basic, a first acidic environment with lower pH is selected. In some embodiments, the first anion is a hexafluorophosphate ion, and the pH of the first acidic environment is 4 to 6, particularly 4.5 to 5.5, particularly 4.8 to 5.2, particularly 4.9 to 5.1, and more particularly about 5.

In some embodiments, the first acidic environment is obtained by adding the first salt to the second solvent. In some embodiments, the first acidic environment is obtained by adding the first salt and an acid consisting of the first anion and a hydrogen ion to the second solvent.

### Formation of the Non-Covalent Bond

A second reactant is added to the first solution, and particularly, is contacted with the protonated first reactant cation in the first solution, in a second acidic environment. Then, the bond is formed, and a second solution that comprises the cation (the first acid salt cation) according to the examples of the present application and the first anion and that is represented by a structure of formula (B1) is obtained.

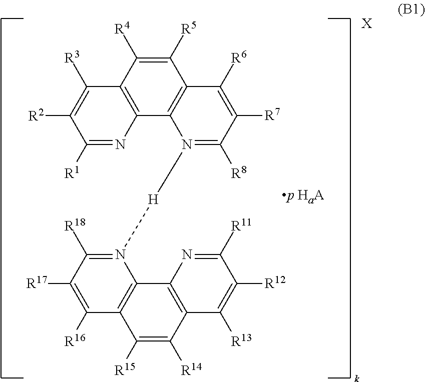

In some embodiments, the second reactant comprises or basically consists of a compound of formula (D), and particularly, consists of the compound. R¹¹, R¹², R¹³, R¹⁴, R¹⁵, R¹⁶, R¹⁷, and R¹⁸ may coincide with the desired cation (formula (A)) or salt (formula (B)) according to the examples of the present application.

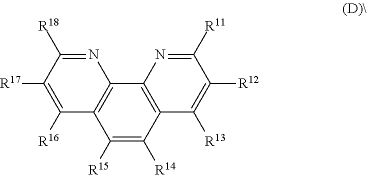

In some embodiments, the second reactant is in excess relative to the protonated first reactant cation. In some embodiments, a molar ratio of the protonated first reactant cation to the second reactant is 1:1 to 1:2, such as about 1:1.0, about 1:1.1, about 1:1.2, about 1:1.3, about 1:1.4, about 1:1.5, about 1:1.6, about 1:1.7, about 1:1.8, about 1:1.9, or about 1:2.0.

In some embodiments, the first solution further comprises a conjugate base of the first acid. In some embodiments, the first solution further comprises the first cation. In some embodiments, the first solution further comprises the second solvent. In some embodiments, the first solution essentially, and particularly, consists of the protonated first acid salt cation of the first reactant, the first cation, the first anion, the conjugate base of the first acid, and the second solvent.

In some embodiments, the second reactant is added to the first solution at a third temperature. In some embodiments, the third temperature is 0° C. to 70° C. In some embodiments, the third temperature is 10° C. to 40° C., more particularly 20° C. to 30° C., and more particularly about 25° C. In some embodiments, the third temperature is 20° C. to 50° C., more particularly 30° C. to 40° C., and more particularly about 37° C. In some embodiments, the third temperature is 0° C. to 20° C., more particularly 0° C. to 10° C., and more particularly about 4° C. In some embodiments, the third temperature is 40° C. to 70° C., more particularly 50° C. to 60° C., and more particularly about 60° C. In some embodiments, the first temperature and the third temperature are the same. In some embodiments, the second temperature and the third temperature are the same. In some embodiments, the first temperature, the second temperature, and the third temperature are the same.

In some embodiments, the pH of the second acidic environment is less than or equal to 6. In some embodiments, the pH of the second acidic environment is 4 to 6, particularly 4.5 to 5.5, particularly 4.8 to 5.2, particularly 4.9 to 5.1, and more particularly about 5. Generally, for the first anion that is less acidic or more basic, a second acidic environment with lower pH is selected. In some embodiments, the first anion is a hexafluorophosphate ion, and the pH of the second acidic environment is 4 to 6, particularly 4.5 to 5.5, particularly 4.8 to 5.2, particularly 4.9 to 5.1, and more particularly about 5. In some embodiments, the pH of the first acidic environment and the pH of the second acidic environment are the same.

In some embodiments, the second acidic environment is obtained by adding the first salt to the second solvent. In some embodiments, the second acidic environment is obtained by adding the first salt and an acid consisting of the first anion and a hydrogen ion to the second solvent. In some embodiments, a reductant is further added to the first solution, and particularly, a reductant is added to the first solution before, after or at the same time as the second reactant is added to the first solution. That is, in some embodiments, the second acidic environment is reductive, and particularly strongly reductive. The non-covalent dimer cation of the present application has strong reducing capacity. Therefore, addition of the reductant is conductive to reduction in a proportion of the non-covalent dimer cation of the present application that is oxidized during reaction, and is conductive to increase in the yield of the salt of the present application. In some embodiments, the reductant includes at least one or more selected from a group consisting of vitamin C, vitamin E, and a derivative of vitamin C or vitamin E.

In some embodiments, the second solution is further purified to obtain a first intermediate including the cation according to the examples of the present application, i.e., the first acid salt cation, and the first anion. In some embodiments, the purification includes dialysis. In some embodiments, the dialysis may be performed through a membrane with molecular weight cut-off (MWCO) of 50 Da to 500 Da, in particular about 100 Da or 200 Da.

### Formation of the Salt of the Present Application

A first base is added to the second solution or contacted with the first intermediate to remove the first acid (HₐA in formula (B1)) in the second solution or the first intermediate. Then, the salt of the present application is obtained.

In some embodiments, the salt of the present application includes the non-covalent dimer cation of the present application and the first anion. For example, the salt of the present application may be of formula (B).

In some embodiments, the first base includes or consists of, but is not limited to, at least one selected from a group consisting of ammonia water, sodium hydroxide, potassium hydroxide, and triethanolamine. Those skilled in the art should understand that any base that can adjust the pH value to be alkaline without causing a further redox reaction can be taken as the first base herein. In some embodiments, the first base is ammonia water, in particular diluted ammonia water. In some embodiments, the first base is sodium hydroxide. In some embodiments, the first base is potassium hydroxide. In some embodiments, the first base is triethanolamine.

In some embodiments, the first base is added to the second solution or contacted with the first intermediate to adjust the pH value of the second solution or a solution including the first intermediate to be alkaline. In some embodiments, the alkalinity refers that the pH value is higher than 8, particularly 8 to 10, particularly 8.5 to 9.5, particularly 8.8 to 9.2, and more particularly about 9.

In some embodiments, contacting the first intermediate with the first base refers that the first intermediate is contacted with the first base in the presence of water.

In some embodiments, after the first base is added to the second solution, purification is further performed to obtain the salt of the present application. In some embodiments, the purification includes dialysis. In some embodiments, the dialysis may be performed through a membrane with molecular weight cut-off (MWCO) of 50 Da to 500 Da, in particular about 100 Da or 200 Da. In some embodiments, the purification includes filtration.

### Use of Protonated Dimer Cation of the Present Application or Salt of the Present Application

In some embodiments, provided is use of the protonated dimer cation of the present application or the salt of the present application in antimicrobials. In some embodiments, provided is use of the protonated dimer cation of the present application or the salt of the present application in the preparation of an antimicrobial agent. In some embodiments, provided is a protonated dimer cation of the present application or a salt of the present application in antimicrobials. In some embodiments, an antimicrobial method, including: applying the protonated dimer cation of the present application or the salt of the present application.

In some embodiments, the microbe is bacteria and/or fungi. In some embodiments, the microbe is bacteria. In some embodiments, the bacteria are gram-positive bacteria. In some embodiments, the bacteria are gram-negative bacteria. In some embodiments, the bacteria are selected from a group consisting of Staphylococcus aureus, Staphylococcus epidermidis, hemolytic staphylococcus, Enterococcus faecalis, Streptococcus mutans, Streptococcus thermophilus, Streptococcus lactis, Streptococcus bovis, Corynebacterium diphtheriae, Listeria monocytogenes, Bacillus subtilis, Bacillus cereus, Clostridium perfringens, Escherichia coli, Salmonella enteritidis, Klebsiella pneumoniae, Proteus vulgaris, Yersinia enterocolitis, Pseudomonas aeruginosa, and Pseudomonas fluorescens. In some embodiments, the bacteria are selected from a group consisting of Staphylococcus aureus, Staphylococcus epidermidis, hemolytic staphylococcus, Enterococcus faecalis, Streptococcus mutans, Streptococcus thermophilus, Streptococcus lactis, Streptococcus bovis, Corynebacterium diphtheriae, Listeria monocytogenes, Bacillus subtilis, Bacillus cereus, and Clostridium perfringens. In some embodiments, the bacteria are selected from a group consisting of Escherichia coli, Salmonella enteritidis, Klebsiella pneumoniae, Proteus vulgaris, Yersinia enterocolitis, Pseudomonas aeruginosa, and Pseudomonas fluorescens. In some embodiments, the bacteria are Staphylococcus aureus. In some embodiments, the bacteria are Staphylococcus epidermidis. In some embodiments, the bacteria are hemolytic staphylococcus. In some embodiments, the bacteria are Enterococcus faecalis. In some embodiments, the bacteria are Streptococcus mutans. In some embodiments, the bacteria are Streptococcus thermophilus. In some embodiments, the bacteria are Streptococcus lactis. In some embodiments, the bacteria are Streptococcus bovis. In some embodiments, the bacteria are Corynebacterium diphtheriae. In some embodiments, the bacteria are Listeria monocytogenes. In some embodiments, the bacteria are Bacillus subtilis. In some embodiments, the bacteria are Bacillus cereus. In some embodiments, the bacteria are Clostridium perfringens. In some embodiments, the bacteria are Escherichia coli. In some embodiments, the bacteria are Salmonella enteritidis. In some embodiments, the bacteria are Klebsiella pneumoniae. In some embodiments, the bacteria are Proteus vulgaris. In some embodiments, the bacteria are Yersinia enterocolitis. In some embodiments, the bacteria are Pseudomonas aeruginosa. In some embodiments, the bacteria are Pseudomonas fluorescens. In some embodiments, the microorganism is fungi. In some embodiments, the fungi are selected from a group consisting of Cryptococcus neoformans, Candida albicans, Trichophyton rubrum, Malassezia globosa, Malassezia furfur, Aspergillus flavus, and Aspergillus niger. In some embodiments, the fungi are Cryptococcus neoformans. In some embodiments, the fungi are Candida albicans. In some embodiments, the fungi are Trichophyton rubrum. In some embodiments, the fungi are Malassezia globosa. In some embodiments, the fungi are Malassezia furfur. In some embodiments, the fungi are Aspergillus flavus. In some embodiments, the fungi are Aspergillus niger.

In some embodiments, provided is use of the protonated dimer cation of the present application or the salt of the present application in the treatment of a tumor. In some embodiments, provided is use of the protonated dimer cation of the present application or the salt of the present application in the preparation of a drug for treating a tumor. In some embodiments, provided is the protonated dimer cation of the present application or the salt of the present application for treating a tumor. In some embodiments, provided is a method for treating a tumor, including: applying the protonated dimer cation of the present application or the salt of the present application.

In some embodiments, the tumor is selected from a group consisting of liver cancer, gastric cancer, colon cancer, bladder cancer, breast cancer, pancreatic cancer, prostate cancer, ovarian cancer, neuroblastoma, glioma, and melanoma. In some embodiments, the tumor is selected from a group consisting of colon cancer, pancreatic cancer, neuroblastoma, and melanoma. In some embodiments, the tumor is liver cancer. In some embodiments, the tumor is gastric cancer. In some embodiments, the tumor is colon cancer. In some embodiments, the tumor is bladder cancer. In some embodiments, the tumor is breast cancer. In some embodiments, the tumor is pancreatic cancer. In some embodiments, the tumor is prostate cancer. In some embodiments, the tumor is ovarian cancer. In some embodiments, the tumor is neuroblastoma. In some embodiments, the tumor is glioma. In some embodiments, the tumor is melanoma.

### EXAMPLES

In the following examples, if a compound of an example included a first reactant, a second reactant, a cation of the present example, or a salt of the present example, and had chirality that was not explicitly specified, the compound may be a chiral mixture such as a racemic mixture.

### Example 1

A salt of Example 1 was specifically shown in formula (1). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

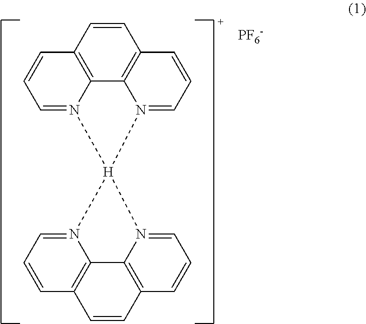

### Preparation Method

A method of preparing of the salt of the present example was as follows. in which:
the first reactant was 1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₇N₄)⁺, m/z=361.4.

An ¹H-NMR spectrum and an HPLC spectrum of the salt of the present example were shown in FIG. 1. HPLC conditions were as follows: a stationary phase of a Shim-pack Scepter C18-120 chromatographic column (4.6× 250 mm, 5 μm); a mobile phase of acetonitrile/20 mM ammonium acetate (25:75, v/v) buffer; a flow rate of 1.0 mL/min; a detection wavelength of 273 nm; a column temperature of 25° C.; and an injection volume of 10 μL.

### Example 2

A salt of Example 2 was specifically shown in formula (2). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=2, and X was a hexafluorosilicate ion.

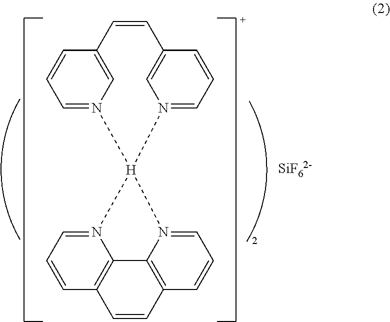

### Preparation Method

A method of preparing of the salt of the present example was as follows. in which:
the first reactant was 1,10-phenanthroline;
the second reactant was 1,10-phenanthroline; and
the first salt was ammonium hexafluorosilicate, i.e., the first anion was a hexafluorosilicate ion.

1 g of the first reactant was placed into 10 mL of 2 M hydrochloric acid at 37° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 37° C. for 12 h to obtain a first aqueous solution including a protonated hydrochloride cation of the first reactant and the first anion.

0.8 g of the second reactant was put into the first aqueous solution at 37° C. and stirred gently for 12 h, to obtain a second aqueous solution including the cation of the present example. A 1 M sodium hydroxide solution was added to adjust the pH value to 9, fully dialyzed with a dialysis bag with a molecular weight cutoff of 200 Da, and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.2 g, and the productivity rate was about 63%.

The salt of the present example was detected by a high-resolution mass spectrometry, to obtain the cation of the present example, (C₂₄H₁₇N₄)+, m/z=361.4.

### Example 3

A salt of Example 3 was specifically shown in formula (3). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=2, and X was a sulfate ion.

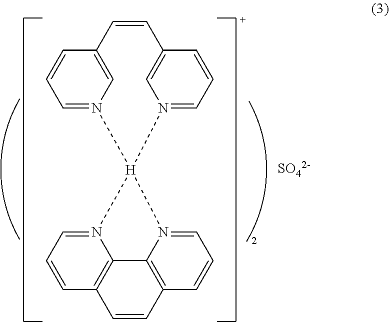

### Preparation Method

A method of preparing of the salt of the present example was as follows. in which:
the first reactant was 1,10-phenanthroline;
the second reactant was 1,10-phenanthroline; and
the first salt was ammonium sulfate, i.e., the first anion was a sulfate ion.

1 g of the first reactant was placed into 10 mL of 2 M hydrochloric acid at 60° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 2 g of the first salt of the present example was added, and the mixture was stirred at 60° C. for 6 h to obtain a first aqueous solution including a protonated hydrochloride cation of the first reactant and the first anion.

0.65 g of the second reactant was put into the first aqueous solution at 60° C. and stirred gently for 36 h, to obtain a second aqueous solution including the cation of the present example. A 1 M sodium hydroxide solution was added to adjust the pH value to 9, fully dialyzed with a dialysis bag with a molecular weight cutoff of 200 Da, and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 0.6 g, and the productivity rate was about 32%.

The salt of the present example was detected by a high-resolution mass spectrometry, to obtain the cation of the present example, (C₂₄H₁₇N₄)⁺, m/z=361.4.

### Example 4

A salt of Example 4 was specifically shown in formula (4). That is, R¹ was methyl, R² was hydrogen, R³ was hydrogen, R⁴ was hydrogen, R⁵ was hydrogen, R⁶ was hydrogen, R⁷ was hydrogen, R⁸ was methyl, R¹¹ was methyl, R¹² was hydrogen, R¹³ was hydrogen, R¹⁴ was hydrogen, R¹⁵ was hydrogen, R¹⁶ was hydrogen, R¹⁷ was hydrogen, R¹⁸ was methyl, k=1, and X was a hexafluorophosphate ion.

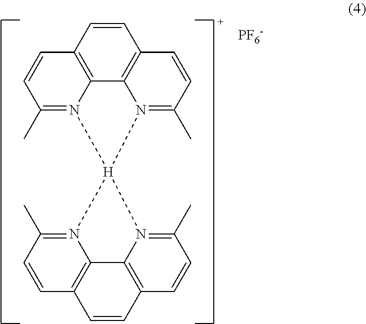

A method of preparing of the salt of the present example was as follows. in which:
the first reactant was 2,9-dimethyl-1,10-phenanthroline;
the second reactant was 2,9-dimethyl-1,10-phenanthroline; and
the first salt was ammonium sulfate, i.e., the first anion was a sulfate ion.

1 g of the first reactant was placed into 10 mL of 2 M hydrochloric acid at 4° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 2 g of the first salt of the present example was added, and the mixture was stirred at 4° C. for 6 h to obtain a first aqueous solution including a protonated hydrochloride cation of the first reactant and the first anion.

0.65 g of the second reactant was put into the first aqueous solution at 4° C. and stirred gently for 48 h, to obtain a second aqueous solution including the cation of the present example. A 1 M sodium hydroxide solution was added to adjust the pH value to 9, fully dialyzed with a dialysis bag with a molecular weight cutoff of 200 Da, and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 64%.

The salt of the present example was detected by a high-resolution mass spectrometry, to obtain the cation of the present example, (C₂₈H₂₅N₄)⁺, m/z=417.5.

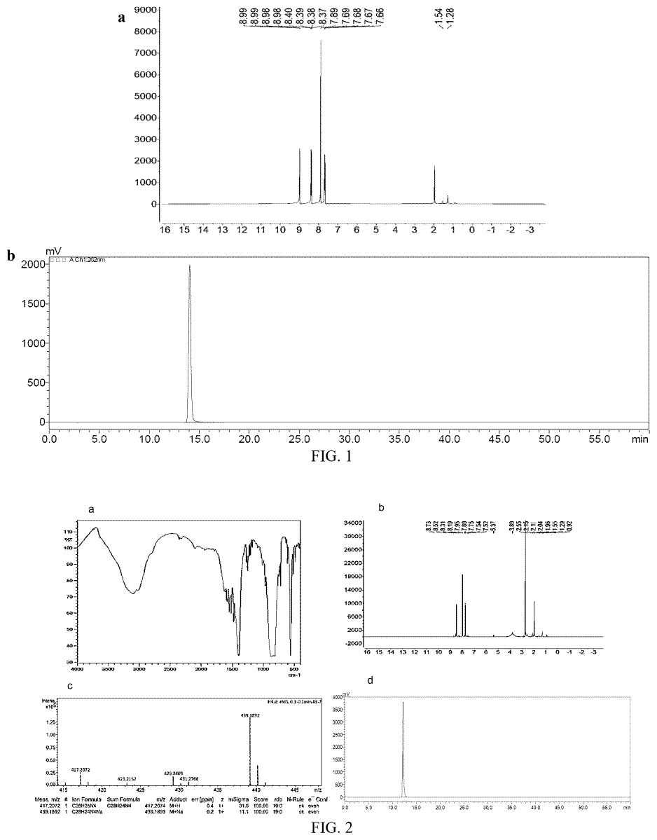

An FTIR spectrum, an ¹H-HRMS spectrum, an HRMS spectrum and an HPLC spectrum of the salt of the present example were shown in FIG. 2. HPLC conditions were as follows: a stationary phase of a Shim-pack Scepter C18-120 chromatographic column (4.6×250 mm, 5 μm); a mobile phase of acetonitrile/water (25:75, v/v) with a pH value of 6.0; a flow rate of 1.0 mL/min; a detection wavelength of 273 nm; a column temperature of 25° C.; and an injection volume of 10 μL.

### Example 5

A salt of Example 5 was specifically shown in formula (5). That is, R¹ was hydrogen, R² was methyl, R³ was methyl, R⁴ was hydrogen, R⁵ was hydrogen, R⁶ was methyl, R⁷ was methyl, R⁸ was hydrogen, R¹¹ was hydrogen, R¹² was methyl, R¹³ was methyl, R¹⁴ was hydrogen, R¹⁵ was hydrogen, R¹⁶ was methyl, R¹⁷ was methyl, R¹⁸ was hydrogen, k=1, and X was a hexafluorophosphate ion.

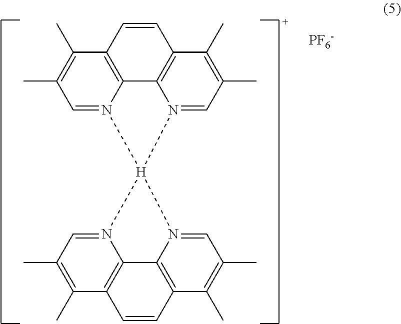

### Preparation Method

A method of preparing of the salt of the present example was as follows. in which:
the first reactant was 3,4,7,8-tetramethyl-1,10-phenanthroline;
the second reactant was 3,4,7,8-tetramethyl-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₃₂H₃₃N₄)⁺, m/z=473.6.

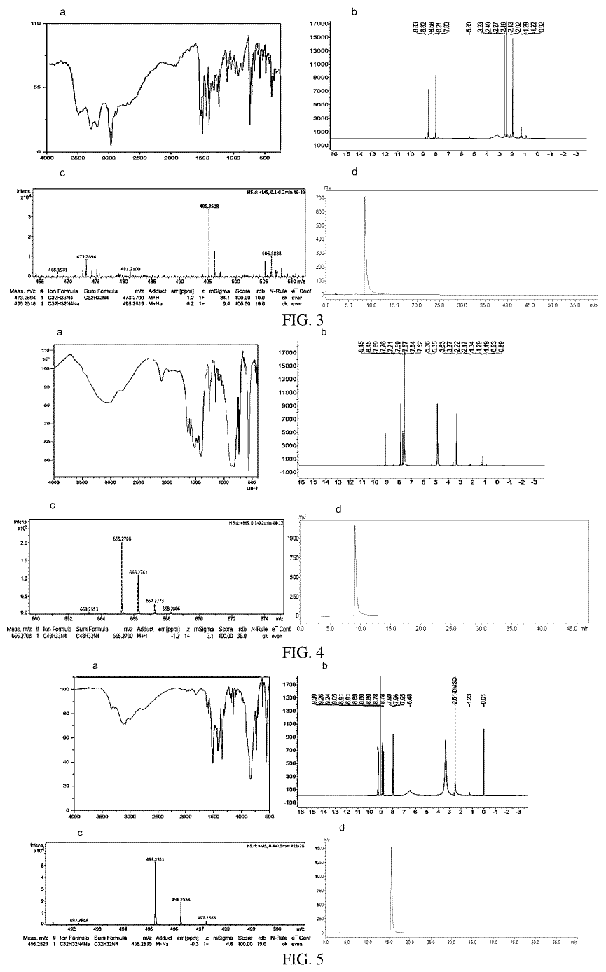

An FTIR spectrum, an 1H-HRMS spectrum, an HRMS spectrum and an HPLC spectrum of the salt of the present example were shown in FIG. 3. HPLC conditions were as follows: a stationary phase of a Shim-pack Scepter C18-120 chromatographic column (4.6×250 mm, 5 μm); a mobile phase of acetonitrile, with a pH value of 6.0; a flow rate of 1.0 mL/min; a detection wavelength of 273 nm; a column temperature of 25° C.; and an injection volume of 10 μL.

### Example 6

A salt of Example 6 was specifically shown in formula (6). That is, R¹ was hydrogen, R² was hydrogen, R³ was phenyl, R⁴ was hydrogen, R⁵ was hydrogen, R⁶ was phenyl, R⁷ was hydrogen, R⁸ was hydrogen, R¹¹ was hydrogen, R¹² was hydrogen, R¹³ was phenyl, R¹⁴ was hydrogen, R¹⁵ was hydrogen, R¹⁶ was phenyl, R¹⁷ was hydrogen, R¹⁸ was hydrogen, k=1, and X was a hexafluorophosphate ion.

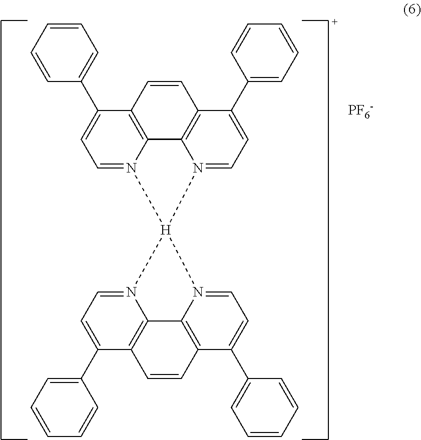

### Preparation Method

A method of preparing of the salt of the present example was as follows. in which:
the first reactant was 4,7-diphenyl-1,10-phenanthroline;
the second reactant was 4,7-dimethyl-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₄₈H₃₇N₄)⁺, m/z=669.8.

An FTIR spectrum, an ¹H-HRMS spectrum, an HRMS spectrum and an HPLC spectrum of the salt of the present example were shown in FIG. 4. HPLC conditions were as follows: a stationary phase of a Shim-pack Scepter C18-120 chromatographic column (4.6×250 mm, 5 μm); a mobile phase of acetonitrile, with a pH value of 6.0; a flow rate of 1.0 mL/min; a detection wavelength of 273 nm; a column temperature of 25° C.; and an injection volume of 10 μL.

### Example 7

A salt of Example 7 was specifically shown in formula (7). That is, R¹ was hydrogen, R² was hydrogen, R³ was hydrogen, R⁴ was nitro, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was nitro, R¹⁵ was hydrogen, R¹⁶ was hydrogen, R¹⁷ was hydrogen, R¹⁸ was hydrogen, k=1, and X was a hexafluorophosphate ion.

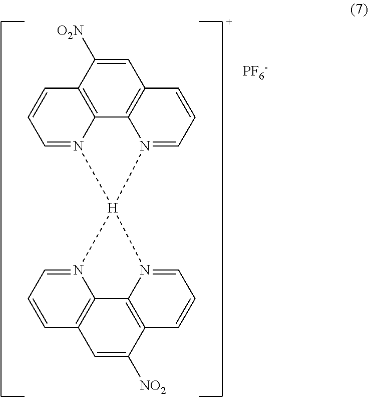

### Preparation Method

A method of preparing of the salt of the present example was as follows. in which:
the first reactant was 5-nitro-1,10-phenanthroline;
the second reactant was 5-nitro-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₅O₄N₆)⁺, m/z=451.4.

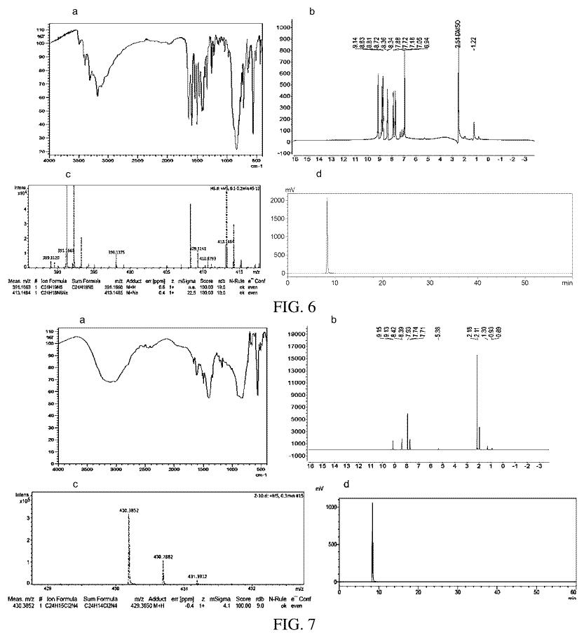

An FTIR spectrum, an ¹H-HRMS spectrum, an HRMS spectrum and an HPLC spectrum of the salt of the present example were shown in FIG. 5. HPLC conditions were as follows: a stationary phase of a Shim-pack Scepter C18-120 chromatographic column (4.6×250 mm, 5 μm); a mobile phase of acetonitrile, with a pH value of 6.0; a flow rate of 1.0 mL/min; a detection wavelength of 273 nm; a column temperature of 25° C.; and an injection volume of 10 μL.

### Example 8

A salt of Example 8 was specifically shown in formula (8). That is, R¹ was hydrogen, R² was hydrogen, R³ was hydrogen, R⁴ was amino, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was amino, R¹⁵ was hydrogen, R¹⁶ was hydrogen, R¹⁷ was hydrogen, R¹⁸ was hydrogen, k=1, and X was a hexafluorophosphate ion.

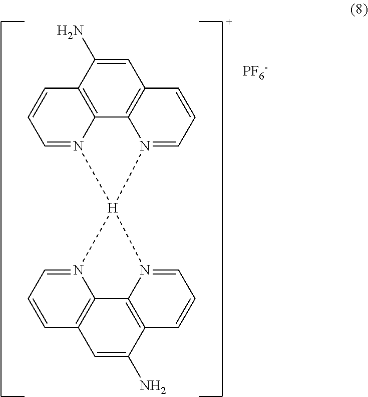

### Preparation Method

A method of preparing of the salt of the present example was as follows. in which:
the first reactant was 5-amino-1,10-phenanthroline;
the second reactant was 5-amino-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₉N₆)⁺, m/z=391.54.

An FTIR spectrum, an ¹H-HRMS spectrum, an HRMS spectrum and an HPLC spectrum of the salt of the present example were shown in FIG. 6. HPLC conditions were as follows: a stationary phase of a Shim-pack Scepter C18-120 chromatographic column (4.6×250 mm, 5 μm); a mobile phase of acetonitrile, with a pH value of 6.0; a pH value was 6.0; a flow rate of 1.0 mL/min; a detection wavelength of 273 nm; a column temperature of 25° C.; and an injection volume of 10 μL.

### Example 9

A salt of Example 9 was specifically shown in formula (9). That is, R¹ was chloro, R² was hydrogen, R³ was hydrogen, R⁴ was hydrogen, R⁵ was hydrogen, R⁶ was hydrogen, R⁷ was hydrogen, R⁸ was hydrogen, R¹¹ was chloro, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

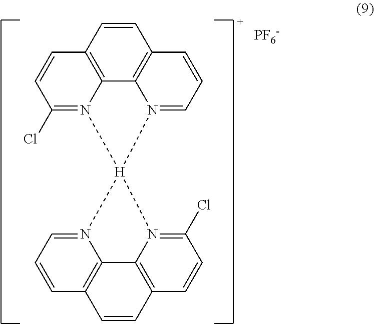

### Preparation Method

A method of preparing of the salt of the present example was as follows. in which:
the first reactant was 2-chloro-1,10-phenanthroline;
the second reactant was 2-chloro-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₅N₄Cl₂)⁺, m/z=430.3.

An FTIR spectrum, an ¹H-HRMS spectrum, an HRMS spectrum and an HPLC spectrum of the salt of the present example were shown in FIG. 7. HPLC conditions were as follows: a stationary phase of a Shim-pack Scepter C18-120 chromatographic column (4.6×250 mm, 5 μm); a mobile phase of acetonitrile/40 mM ammonium acetate (20:80, v/v) buffer, with a pH value of 6.8; a flow rate of 1.0 mL/min; a detection wavelength of 273 nm; a column temperature of 25° C.; and an injection volume of 10 μL.

### Example 10

A salt of Example 10 was specifically shown in formula (10). That is, R¹ was cyano, R² was hydrogen, R³ was hydrogen, R⁴ was hydrogen, R⁵ was hydrogen, R⁶ was hydrogen, R⁷ was hydrogen, R⁸ was hydrogen, R¹¹ was cyano, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

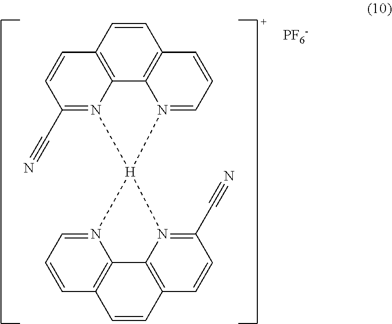

### Preparation Method

A method of preparing of the salt of the present example was as follows. in which:
the first reactant was 2-cyano-1,10-phenanthroline;
the second reactant was 2-cyano-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₆H₁₅N₆)⁺, m/z=411.4.

### Example 11

A salt of Example 11 was specifically shown in formula (11). That is, R¹ was hydrogen R² was hydrogen, R³ was hydrogen, R⁴ was hydrogen, R⁵ was hydrogen, R⁶ was hydrogen, R⁷ was hydrogen, R⁸ was hydrogen, R¹¹ was methyl, R¹² was hydrogen, R¹³ was hydrogen, R¹⁴ was hydrogen, R¹⁵ was hydrogen, R¹⁶ was hydrogen, R¹⁷ was hydrogen, R¹⁸ was methyl, k=1, and X was a hexafluorophosphate ion.

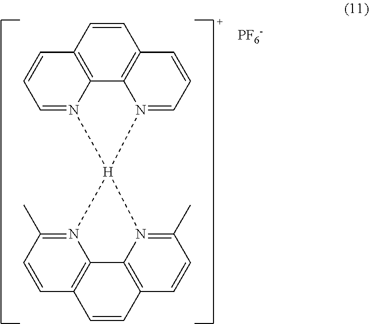

### Preparation Method

A method of preparing of the salt of the present example was as follows. in which:
the first reactant was 1,10-phenanthroline;
the second reactant was 2,9-dimethyl-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₆H₂₁N₄)⁺, m/z=389.5.

### Example 12

A salt of Example 12 was specifically shown in formula (12). That is, R¹ was methyl, R² was hydrogen, R³ was hydrogen, R⁴ was hydrogen, R⁵ was hydrogen, R⁶ was hydrogen, R⁷ was hydrogen, R⁸ was methyl, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

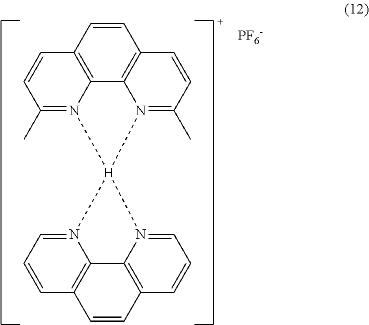

### Preparation Method

A method of preparing of the salt of the present example was as follows. in which:
the first reactant was 2,9-dimethyl-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₆H₂₁N₄)⁺, m/z=389.5.

### Example 13

A salt of Example 13 was specifically shown in formula (13). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was methyl, R¹³ was methyl, R¹⁴ was hydrogen, R¹⁵ was hydrogen, R¹⁶ was methyl, R¹⁷ was methyl, R¹⁸ was hydrogen, k=1, and X was a hexafluorophosphate ion.

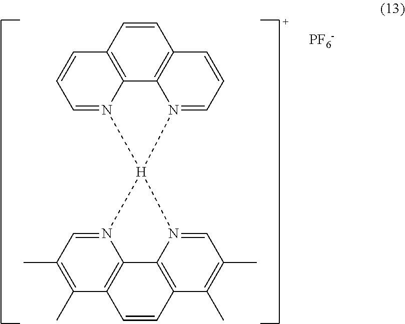

### Preparation Method

A method of preparing of the salt of the present example was as follows. in which:
the first reactant was 1,10-phenanthroline;
the second reactant was 3,4,7,8-tetramethyl-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₈H₂₅N₄)⁺, m/z=417.5.

### Example 14

A salt of Example 14 was specifically shown in formula (14). That is, R¹ was hydrogen, R² was methyl, R³ was methyl, R⁴ was hydrogen, R⁵ was hydrogen, R⁶ was methyl, R⁷ was methyl, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

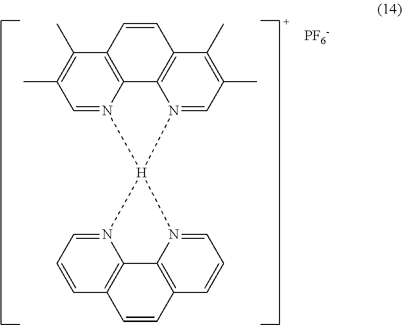

### Preparation Method

A method of preparing of the salt of the present example was as follows. in which:
the first reactant was 3,4,7,8-tetramethyl-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₈H₂₅N₄)⁺, m/z=417.5.

### Example 15

A salt of Example 15 was specifically shown in formula (15). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was phenyl, R¹⁴ was hydrogen, R¹⁵ was hydrogen, R¹⁶ was phenyl, R¹⁷ was hydrogen, R¹⁸ was hydrogen, k=1, and X was a hexafluorophosphate ion.

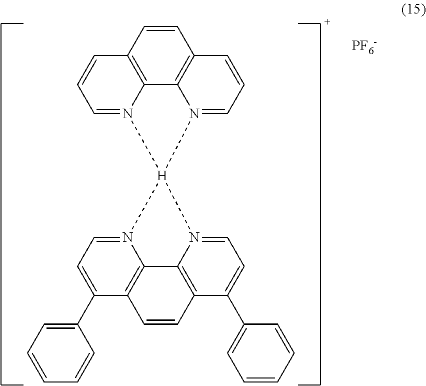

### Preparation Method

A method of preparing of the salt of the present example was as follows. in which:
the first reactant was 1,10-phenanthroline;
the second reactant was 4,7-dimethyl-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₃₆H₂₇N₄)⁺, m/z=515.6.

### Example 16

A salt of Example 16 was specifically shown in formula (16). That is, R¹ was hydrogen, R² was hydrogen, R³ was phenyl, R⁴ was hydrogen, R⁵ was hydrogen, R⁶ was phenyl, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

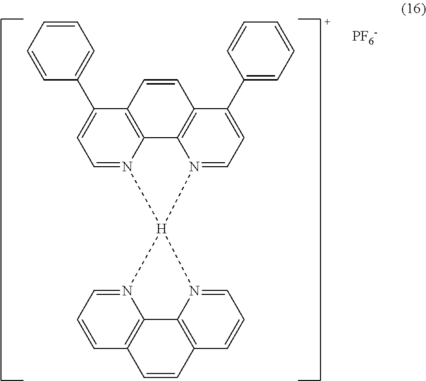

### Preparation Method

A method of preparing of the salt of the present example was as follows. in which:
the first reactant was 4,7-diphenyl-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₃₆H₂₇N₄)⁺, m/z=515.6.

### Example 17

A salt of Example 17 was specifically shown in formula (17). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was nitro, R¹⁵ was hydrogen, R¹⁶ was hydrogen, R¹⁷ was hydrogen, R¹⁸ was hydrogen, k=1, and X was a hexafluorophosphate ion.

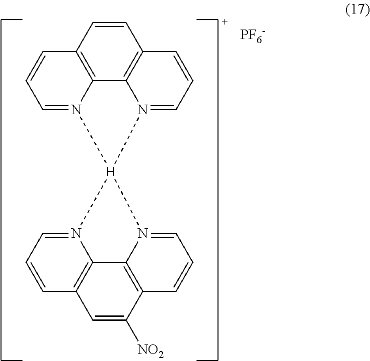

### Preparation Method

A method of preparing of the salt of the present example was as follows. in which:
the first reactant was 1,10-phenanthroline;
the second reactant was 4-nitro-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₅O₂N₅)⁺, m/z=405.4.

### Example 18

A salt of Example 18 was specifically shown in formula (18). That is, R¹ was hydrogen, R² was hydrogen, R³ was hydrogen, R⁴ was nitro, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

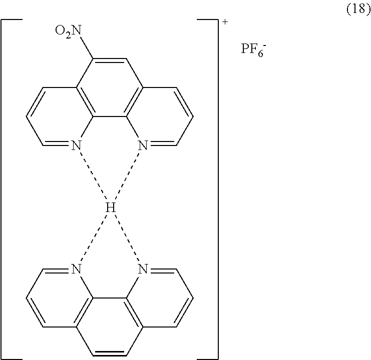

### Preparation Method

A method of preparing of the salt of the present example was as follows. in which:
the first reactant was 4-nitro-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₅O₂N₅)⁺, m/z=405.4.

### Example 19

A salt of Example 19 was specifically shown in formula (19). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was amino, R¹⁵ was hydrogen, R¹⁶ was hydrogen, R¹⁷ was hydrogen, R¹⁸ was hydrogen, k=1, and X was a hexafluorophosphate ion.

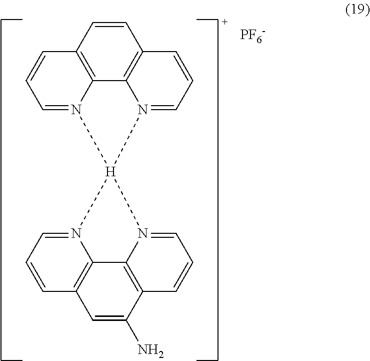

### Preparation Method

A method of preparing of the salt of the present example was as follows. in which:
the first reactant was 1,10-phenanthroline;
the second reactant was 4-amino-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₈N₅)⁺, m/z=376.4.

### Example 20

A salt of Example 20 was specifically shown in formula (20). That is, R¹ was hydrogen, R² was hydrogen, R³ was hydrogen, R⁴ was amino, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

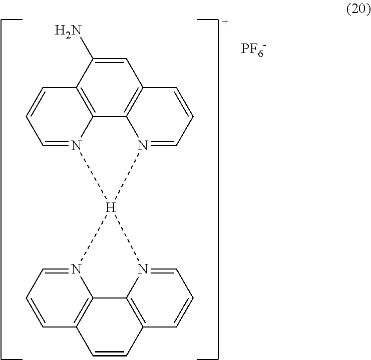

### Preparation Method

A method of preparing of the salt of the present example was as follows. in which:
the first reactant was 4-amino-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₈N₅)⁺, m/z=376.4.

### Example 21

A salt of Example 21 was specifically shown in formula (21). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was chloro, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

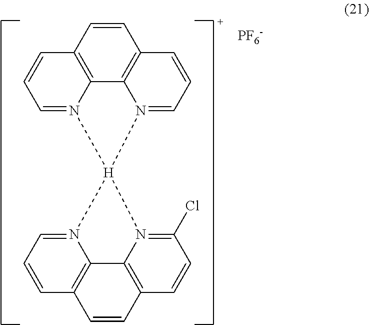

### Preparation Method

A method of preparing of the salt of the present example was as follows. in which:
the first reactant was 1,10-phenanthroline;
the second reactant was 2-chloro-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₆N₄Cl)⁺, m/z=395.8.

### Example 22

A salt of Example 22 was specifically shown in formula (22). That is, R¹ was chloro, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

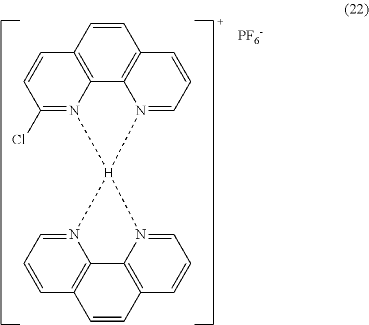

### Preparation Method

A method of preparing of the salt of the present example was as follows. in which:
the first reactant was 2-chloro-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₄H₁₆N₄Cl)⁺, m/z=395.8.

### Example 23

A salt of Example 23 was specifically shown in formula (23). That is, R¹ was hydrogen, R² was hydrogen, R³ was hydrogen, R⁴ was hydrogen, R⁵ was hydrogen, R⁶ was hydrogen, R⁷ was hydrogen, R⁸ was hydrogen, R¹¹ was cyano, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

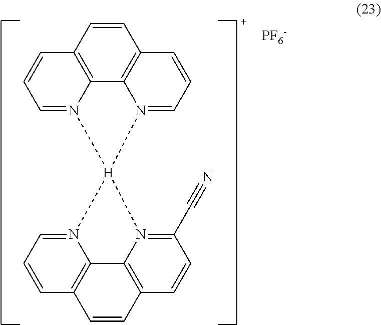

### Preparation Method

A method of preparing of the salt of the present example was as follows. in which:
the first reactant was 1,10-phenanthroline;
the second reactant was 2-cyano-1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₅H₁₆N₅)⁺, m/z=386.4.

### Example 24

A salt of Example 24 was specifically shown in formula (24). That is, R¹ was cyano, R² was hydrogen, R³ was hydrogen, R⁴ was hydrogen, R⁵ was hydrogen, R⁶ was hydrogen, R⁷ was hydrogen, R⁸ was hydrogen, R¹¹ was hydrogen, R¹² was hydrogen, R¹³ was hydrogen, R¹⁴ was hydrogen, R¹⁵ was hydrogen, R¹⁶ was hydrogen, R¹⁷ was hydrogen, R¹⁸ was hydrogen, k=1, and X was a hexafluorophosphate ion.

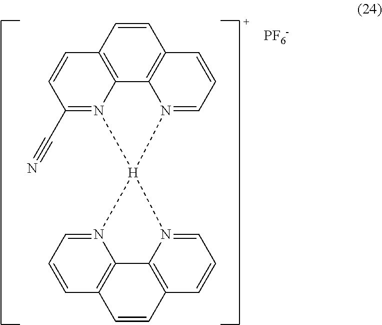

### Preparation Method

A method of preparing of the salt of the present example was as follows. in which:
the first reactant was 2-cyano-1,10-phenanthroline;
the second reactant was 1,10-phenanthroline; and
the first salt was ammonium hexafluorophosphate, i.e., the first anion was a hexafluorophosphate ion.

1 g of the first reactant was placed into 10 mL of 2 M hydrochloric acid at 25° C. and stirred intensely until the first reactant was completely dissolved. The solution was heated to 85° C. and maintained at the temperature for 30 min, and concentrated under reduced pressure to 2 mL. After the solution was cooled, crystals were precipitated, washed 3 times with an ethanol solution, and dried to obtain a first reactant hydrochloride.

0.8 g of the first reactant hydrochloride was placed into 10 mL of ultrapure water and stirred intensely until the first reactant hydrochloride was completely dissolved, 4 g of the first salt of the present example was added, and the mixture was stirred at 25° C. for 6 h to obtain a first aqueous solution including a protonated hydrochloride cation of the first reactant and the first anion.

0.8 g of the second reactant was put into the first aqueous solution at 25° C. and stirred gently for 24 h, to obtain a second aqueous solution including the cation of the present example. The second aqueous solution was completely dialyzed with a dialysis bag with molecular weight cut-off of 200 Da, and then dried to obtain a first intermediate.

The first intermediate was dissolved in 10 mL of ultrapure water, and added with diluted ammonia water to adjust the pH to 9 till a large amount of white precipitates were precipitated, followed by filtration; and the filter cake was washed twice with water and dried to obtain a product, i.e., the salt of the present example.

### Detection of the Product or the Intermediate

The first reactant hydrochloride in the present example was white crystals, with a yield of about 0.8 g and a productivity rate of about 80%.

The yield of the salt of the present example was about 1.6 g, and the productivity rate was about 85%.

The salt of the present example was detected by a high-resolution mass spectrometry, to obtain the cation of the present example, (C₂₅H₁₆N₅)⁺, m/z=386.4.

### Stability Test

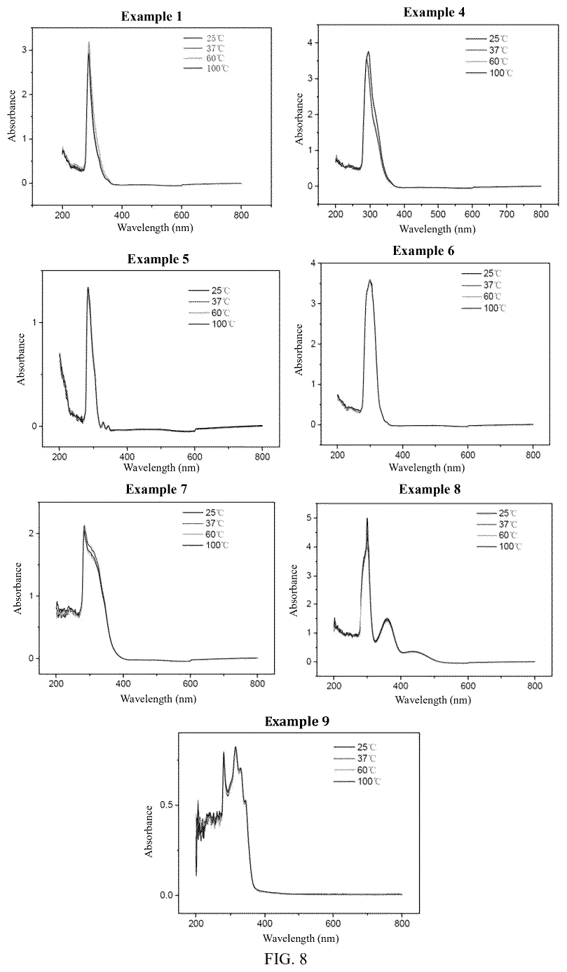

The salts of Examples 1, 4, 5, 6, 7, 8, and 9 were respectively prepared into aqueous solutions, and UV-Vis absorption spectra of the aqueous solutions were measured at different temperatures (25° C., 37° C., 60° C., and 100° C.) to determine their stability. The results were shown in FIG. 8. The results showed that the aqueous solutions of the salts of these examples had little changes in UV-Vis absorption spectra at different temperatures, indicating that they had very high thermal stability within the tested temperature range.

Further stability studies had shown that the compounds of the examples and their solutions (such as aqueous solutions, ethanol solutions, or acetonitrile solutions) could still maintain their stability when placed at room temperature for 1 year or more, and even heated to more than 100° C.

### Anti-Tumor Effects of Compounds of the Examples at Different Concentrations

Examples 1, 5 and 8 were taken as experimental group compounds, a blank control group (no medicament added) was set, and the anti-tumor abilities of the compounds in the present application at a cellular level were detected.

The following various tumor cells were inoculated evenly into a 96-well plate, with about 5,000 cells per well, and the cells were cultured using a medium (see parentheses) for 6 h.
human gastric adenocarcinoma cells AGS (F12)
human gastric adenocarcinoma cells MKN-45 (RPMI1640)
human colon cancer cells SW-480 (L15)
human liver cancer cells HepG2
human liver cancer cells SMMC-7721
human liver cancer cells HuH-7
human neuroblastoma cells SH (DMEM/F12)
mouse melanoma cells B16 (RPMI1640)
human pancreatic cancer cells PANC-1 (DMEM HG)
human breast cancer cells MCF-7 (DMEM HG)
human breast cancer cells BT-549 (DMEM HG)
human prostate cancer cells 22RV1 (RPMI1640)
human gastric adenocarcinoma cells HGC-27 (RPMI1640)
human colon cancer cells HCT-116 (McCoy's 5A)
human liver cancer cells SK-Hep-1
human liver cancer cells Hep3B
human liver cancer cells LC/PRF/5
human liver cancer cells A549 (DMEM HG)
human glioma cells U251 (DMEM HG)
mouse liver cancer cells H22 (RPMI1640)
human pancreatic cancer cells BxPC3 (DMEM HG)
human breast cancer cells SKBR3 (DMEM HG)
human bladder cancer cells 5637 (RPMI1640)
human ovarian cancer cells Anglne (DMEM HG)

After the cells were completely adherent, all the medium was removed. 100 μL of cell culture medium corresponding to a serum-free medium was added to each well. Meanwhile, different concentrations of example solutions were added to each well according to the groups till the final concentrations of the experimental groups were respectively 0.00625 mg/mL, 0.0125 mg/mL, 0.025 mg/mL, 0.05 mg/mL, 0.1 mg/mL, and 0.2 mg/mL.

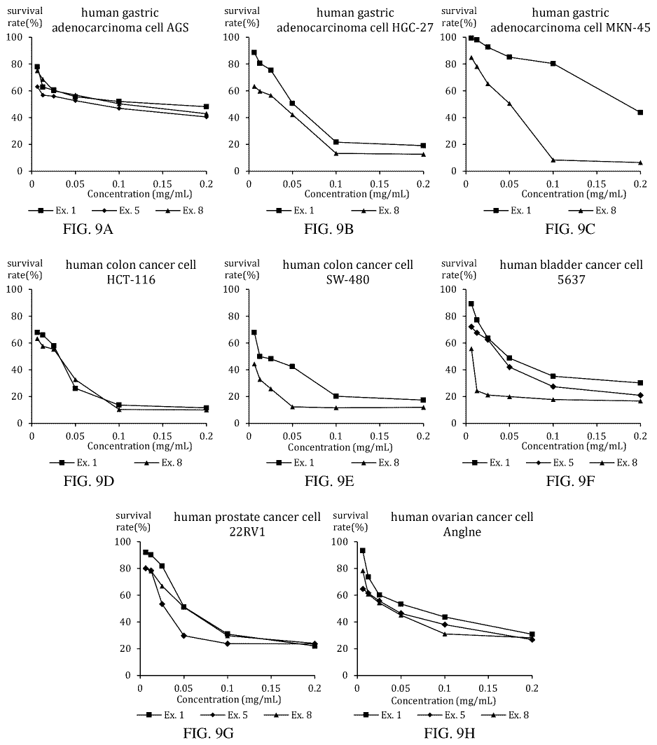

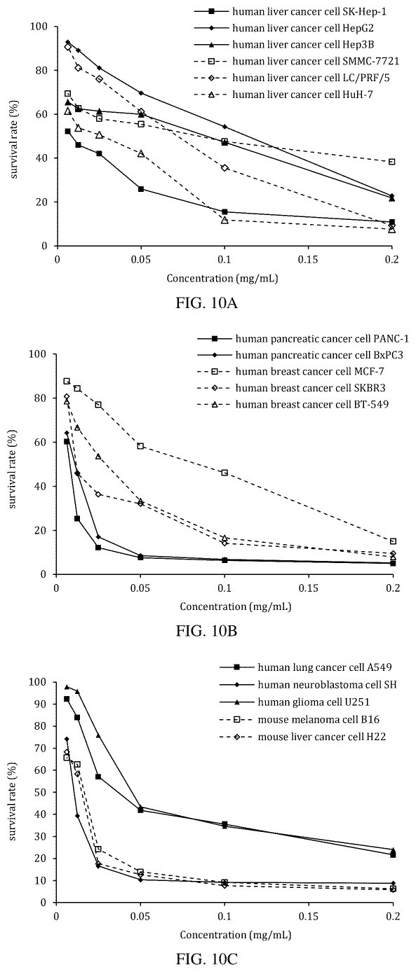

After 24 h of culture, the cell viability was measured by CCK-8 assay. After all the medium was removed, 90 μL of the corresponding medium+10 μL of CCK-8 detection agent were added to each well. An absorbance value at a wavelength of 450 nm was detected after 1 h of incubation. The results were shown in FIGS. 9A-9H and 10A-10C. It can be seen that the tumor cell viability decreased with the increase in concentration after the salt of the present application was added to the cell culture solution. Specifically, it can be seen from FIGS. 9A-9H and 10A-10C that the cation of each example of the present application had a significant anti-tumor effect on various tumors, such as liver cancer, gastric cancer, colon cancer, bladder cancer, breast cancer, pancreatic cancer, prostate cancer, ovarian cancer, neuroblastoma, glioma, and melanoma, and for example, had excellent anti-tumor effects at very low concentrations (e.g., had a survival rate less than 20% at 0.05 mg/mL) for colon cancer, pancreatic cancer, neuroblastoma, melanoma, etc.

### Cytotoxicity Test

Example 1 was taken as the compound of experimental group, a blank control group (free of a drug) was set, and the cytotoxicity of the compound of the present application at a cellular level was detected.

Normal human cells (human normal hepatocytes L02, and human umbilical vein endothelial cells HUVEC) were inoculated evenly into a 96-well plate, with about 5000 cells per well, and cultured for 6 h. After the cells were completely adherent, all the medium was removed. 100 μL of cell culture medium corresponding to a serum-free medium was added to each well. Meanwhile, different concentrations of example solutions were added to each well according to the groups till the final concentrations of the experimental groups were respectively 0.00625 mg/mL, 0.0125 mg/mL, 0.025 mg/mL, 0.05 mg/mL, 0.1 mg/mL, and 0.2 mg/mL.

After 24 h of culture, the cell viability was measured using CCK-8. After all the medium was removed, 90 μL of the corresponding medium+10 μL of CCK-8 detection agent were added to each well. An absorbance value at a wavelength of 450 nm was detected after 1 h of incubation. The results were shown in FIG. 11. It can be seen that the viability of normal human cells was almost not affected after the salt of the present application was added to the cell culture solution.

It was well known that 1,10-phenanthroline and the analogues thereof were compounds with acute toxicity to animals, and had a toxicological mechanism in that phenanthroline, as an active metal chelator, bound to metal ions in organisms to produce severe toxicity. In contrast, the cation of the present application had no significant toxicity to normal cells and animals, and had the biological safety that allowed it to be applied in vivo.

### IC50 Determination

The cells were cultured in a 96-well plate for 24 h and then treated with the salt of Examples 1, 4, 5, 6, 7, 8, or 9 for 8 h. The cells were washed with PBS (pH 7.4), added with a CCK-8 solution (Beijing Beyotime Biotechnology) to each well according to the instructions, and then incubated for 4 h. An absorbance value at a wavelength of 570 nm was detected using a microplate reader (Bio-Rad, USA), and IC50 was calculated. The results were shown in Table 1.

TABLE 1
IC50 (μmol/mL) of various cells for salts of various examples

| Cell line (medium) | Ex. 1 | Ex. 4 | Ex. 5 | Ex. 6 | Ex. 7 | Ex. 8 | Ex. 9 |
|---|---|---|---|---|---|---|---|
| Mouse liver cancer H22 cell line (RPMI 1640) | 0.052 | 0.042 | 0.086 | 0.56 | 0.32 | 0.052 | 0.56 |
| Mouse melanoma B16 cell line (RPMI 1640) | 0.18 | 0.082 | 0.32 | 0.56 | 0.32 | 0.28 | 0.56 |
| Human lung adenocarcinoma A549 cell line (DMEM HG) | 0.30 | 0.16 | 0.36 | 0.62 | 0.32 | 0.18 | 0.56 |
| Human neuroglioma U521MG cell Line (DMEM HG) | 0.56 | 0.35 | 0.50 | 0.56 | 0.32 | 0.56 | 0.56 |
| Human neuroblastoma SH-SY5Y cell line (DMEM/F12) | 0.028 | 0.020 | 0.032 | 0.36 | 0.032 | 0.032 | 0.032 |

It can be seen that the salts of the examples had certain inhibition effects on tumor cell proliferation for these tumor cell lines. The performances of Example 1 and Example 4 were the most prominent. Especially for H22 cells and SH-SY5Y cells, the IC50 of Example 1 and Example 4 was relatively lower than that of other compounds.

### Observation of Cell Apoptosis

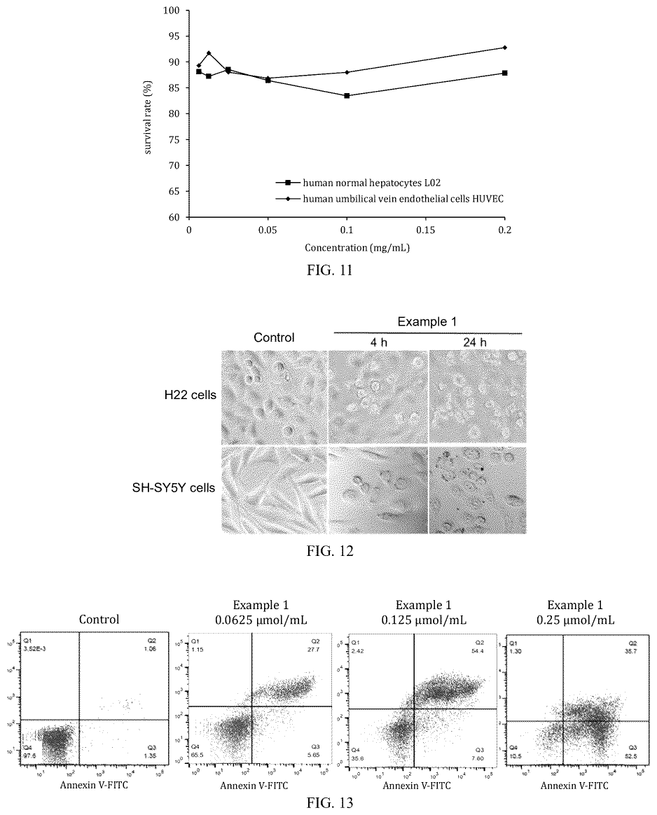

FIG. 12 showed the results after H22 cells and SH-SY5Y cells were treated with the salt of Example 1 at a concentration of 0.00625 μmol/mL for 4 h and 24 h. It can be seen that with the extension of the culture time of the salt of Example 1, the apoptosis was more significant, and the cells showed apoptotic bodies.

FIG. 13 showed the results of the treatment of H22 cells with control group cells and the salt of Example 1 which had concentrations of 0.0625 μmol/mL, 0.125 μmol/mL and 0.25 μmol/mL respectively for 8 h, and the evaluation on the apoptosis by a flow cytometer (BD Biosciences, USA) using Annexin V-FITC. It can be seen that the number of apoptotic cells increased with the increase in the concentration of the salt of Example 1. As shown in FIG. 12, many H22 cells treated with Example 1 showed apoptosis, indicating that an inhibition mechanism of the cation of the example in the present application to cell proliferation may be related to the enhancement of apoptosis in tumor cells.

### Tests for MIC, MBC and MFC

The antimicrobial activity of the compound of the present application was detected by the tests for the minimal inhibitory concentration (MIC) and the minimal bactericide concentration (MBC) or the minimal fungicidal concentration (MFC).

Bacteria and fungi were counted and then inoculated evenly into a 96-well plate, with about 100 bacterial or fungal cells per well. 100 μL of total nutrient SDA medium was used as the medium, and 20 μL of paraffin oil was added for blocking in the case of anaerobic bacteria. At the same time, the solutions of salts of Examples 1-9 with different concentrations were added to each well according to groups, so that the final concentrations of experimental groups were 0.5 mg/mL, 1 mg/mL, 1.5 mg/mL, 2 mg/mL, 2.5 mg/mL, 3 mg/mL, 3.5 mg/mL, 4 mg/mL, 4 mg/mL, 4.5 mg/mL, 5 mg/mL, 5.5 mg/mL, and 6 mg/mL.

After 12 h of culture, the absorbance value at a wavelength of 600 nm was detected, followed by MIC determination.

Subsequently, 20 μL of bacterial solution was extracted from each well and added to a brand-new 96-well plate; 180 μL of total nutrient SDA medium was added, and continued to culture for 12 h; and the absorbance value at a wavelength of 600 nm was detected, followed by MBC or MFC determination.

The results of a bacteria groups were shown in Table 2, and the results of a fungi groups were shown in Table 3. It can be seen that the cation of the example in the present application had low MIC and MBC for various gram-positive bacteria and gram-negative bacteria, that is, had strong ability to inhibit bacteria. The cation of the example in the present application had low MIC and MFC for various fungi, that is, had strong ability to inhibit fungi.

TABLE 2
Results of antibacterial test for example compounds

| | | Example 1 | | Example 2 | | Example 3 | | Example 4 | | Example 5 | |
|---|---|---|---|---|---|---|---|---|---|---|---|
| | | MIC | MBC | MIC | MBC | MIC | MBC | MIC | MBC | MIC | MBC |
| Gram- | S. aureus | 0.5 | 3 | 0.4 | 2.5 | 0.5 | 3 | 0.4 | 2.5 | — | — |
| positive | S. epidermidis | 0.5 | 3 | 0.4 | 2.5 | 0.5 | 3 | 0.4 | 2.5 | — | — |
| bacteria | H. staphylococcus | 0.2 | 0.6 | 0.2 | 0.5 | 0.2 | 0.6 | 0.2 | 0.5 | — | — |
| | E. faecalis | 8 | 30 | 6 | 20 | 8 | 30 | 2 | 10 | — | — |
| | S. mutans | 1 | 5 | 0.8 | 4 | 1 | 5 | 0.8 | 4 | — | — |
| | S. thermophilus | 1 | 5 | 0.8 | 4 | 1 | 5 | 0.8 | 4 | — | — |
| | S. lactis | 1.5 | 6 | 1.2 | 4 | 1.5 | 6 | 1.2 | 4 | 3 | 12 |
| | S. bovis | 0.8 | 3 | 0.6 | 2 | 0.8 | 3 | 0.6 | 2 | — | — |
| | C. diphtheriae | 2 | 6 | 1.6 | 5 | 2 | 6 | 1.6 | 5 | — | — |
| | L. monocytogenes | 0.3 | 1.5 | 0.3 | 1.2 | 0.3 | 1.5 | 0.3 | 1.2 | — | — |
| | B. subtilis | 0.6 | 3 | 0.6 | 2.5 | 0.6 | 3 | 0.6 | 2.5 | — | — |
| | B. cereus | 2 | 9 | 2 | 6 | 2 | 9 | 1 | 3 | — | — |
| | C. perfringens | 3 | 12 | 2.5 | 9 | 3 | 12 | 1.2 | 6 | — | — |
| Gram- | E. coli | 0.5 | 3 | 0.4 | 3 | 0.5 | 3 | 0.4 | 2 | — | — |
| negative | S. enteritidis | 1 | 5 | 0.8 | 4 | 1 | 5 | 0.5 | 3 | — | — |
| bacteria | K. pneumoniae | 1 | 6 | 0.8 | 5 | 1 | 6 | 0.5 | 3 | 4 | 12 |
| | P. vulgaris | 1 | 6 | 0.8 | 4 | 1 | 6 | 0.6 | 4 | — | — |
| | Y. enterocolitis | 0.5 | 3 | 0.4 | 2 | 0.5 | 3 | 0.2 | 2.5 | — | — |
| | P. aeruginosa | 0.5 | 3 | 0.4 | 2 | 0.5 | 3 | 0.4 | 1.5 | — | — |
| | P. fluorescens | 0.5 | 3 | 0.4 | 3 | 0.5 | 3 | 0.3 | 2 | — | — |

| | | Example 6 | | Example 7 | | Example 8 | | Example 9 | |
|---|---|---|---|---|---|---|---|---|---|
| | | MIC | MBC | MIC | MBC | MIC | MBC | MIC | MBC |
| Gram- | S. aureus | — | — | — | — | 3 | 15 | — | — |
| positive | S. epidermidis | — | — | — | — | 3 | 15 | — | — |
| bacteria | H. staphylococcus | — | — | — | — | 5 | 20 | — | — |
| | E. faecalis | 12 | 20 | — | — | 60 | — | — | — |
| | S. mutans | — | — | — | — | 30 | — | — | — |
| | S. thermophilus | — | — | — | — | 20 | 100 | — | — |
| | S. lactis | — | — | — | — | 30 | — | — | — |
| | S. bovis | — | — | — | — | 3 | 20 | — | — |
| | C. diphtheriae | — | — | — | — | 6 | 25 | — | — |
| | L. monocytogenes | — | — | — | — | 5 | 20 | — | — |
| | B. subtilis | — | — | — | — | 7 | 25 | — | — |
| | B. cereus | — | — | 0.5 | 4 | 9 | 35 | 2 | 10 |
| | C. perfringens | — | — | — | — | 12 | — | — | — |
| Gram- | E. coli | — | — | — | — | 6 | 24 | — | — |
| negative | S. enteritidis | — | — | — | — | 9 | 35 | — | — |
| bacteria | K. pneumoniae | — | — | — | — | 12 | — | — | — |
| | P. vulgaris | — | — | — | — | 12 | — | — | — |
| | Y. enterocolitis | — | — | — | — | 6 | 30 | — | — |
| | P. aeruginosa | — | — | — | — | 6 | 20 | — | — |
| | P. fluorescens | — | — | — | — | 6 | 20 | — | — |

“—”: No test was conducted.

TABLE 3
Results of anti-fungal test for example compounds

| | Example 1 | | Example 2 | | Example 3 | | Example 4 | |
|---|---|---|---|---|---|---|---|---|
| | MIC | MFC | MIC | MFC | MIC | MFC | MIC | MFC |
| C. neoformans | 0.5 | 6 | 0.4 | 5 | 0.5 | 6 | 0.5 | 4 |
| C. albicans | 0.5 | 9 | 0.4 | 8 | 0.5 | 9 | 0.5 | 6 |
| T. rubrum | 2 | 12 | 1.6 | 10 | 2 | 12 | 1.5 | 6 |
| M. globosa | 2 | 12 | 2 | 10 | 2 | 12 | 2 | 8 |
| M. furfur | 1 | 6 | 1 | 6 | 1 | 6 | 1 | 4 |
| A. flavus | 0.5 | 6 | 0.4 | 6 | 0.5 | 6 | 0.3 | 5 |
| A. niger | 0.5 | 6 | 0.4 | 4 | 0.5 | 6 | 0.5 | 4 |

### Disk Diffusion Antibacterial Test

The salts of Examples 1, 4, 5 and 6 were used as medicaments of experimental groups, cefalexin and streptomycin were used as control groups, and DMSO was used as the negative control.

All instruments were autoclaved at 121° C. for 30 min for sterilization. Staphylococcus aureus, Escherichia coli, β-hemolytic Streptococcus, Streptococcus pneumoniae, Proteus vulgaris, and Pseudomonas syringae pv. tabaci grew overnight in a Luria-Bertani (LB) liquid medium and collected during an exponential growth phase. 100 μL of bacterial solution at a concentration of 10⁶cfu/mL was added to an LB agar plate, and then, standard filter paper loaded with 20 μg of drug (the salts of the examples, or cefalexin or streptomycin of the control groups) was placed on agar. After 24 h of culture at 37° C., the diameters of inhibition zones were recorded, respectively.

The results were shown in FIG. 14A and FIG. 14B. It can be seen that the salt of Example 1 had an excellent antibacterial effect, and was significantly superior to the control group and Examples 4, 5 and 6 in the diameter of the inhibition zone.

### Disk Diffusion Antifungal Test

The salts of Examples 1, 4, 5 and 6 were used as medicaments of experimental groups, fluconazole (FLC) was used as the control group, and DMSO was used as the negative control.

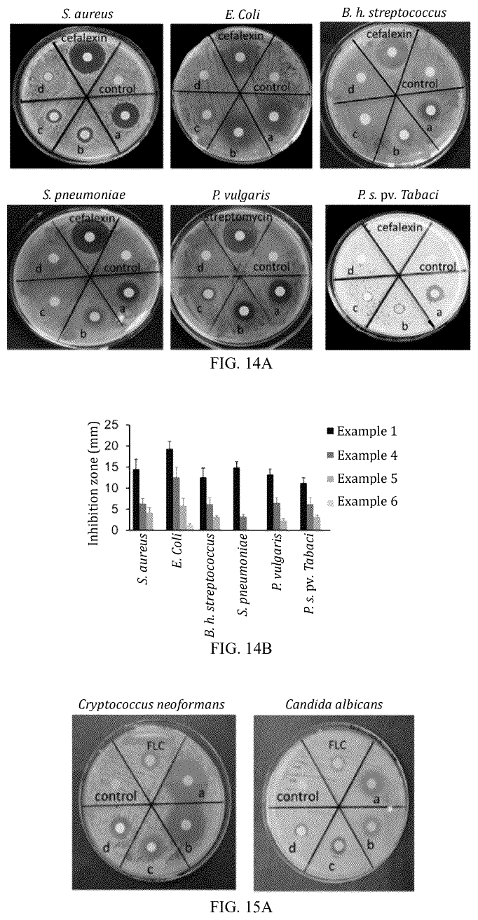

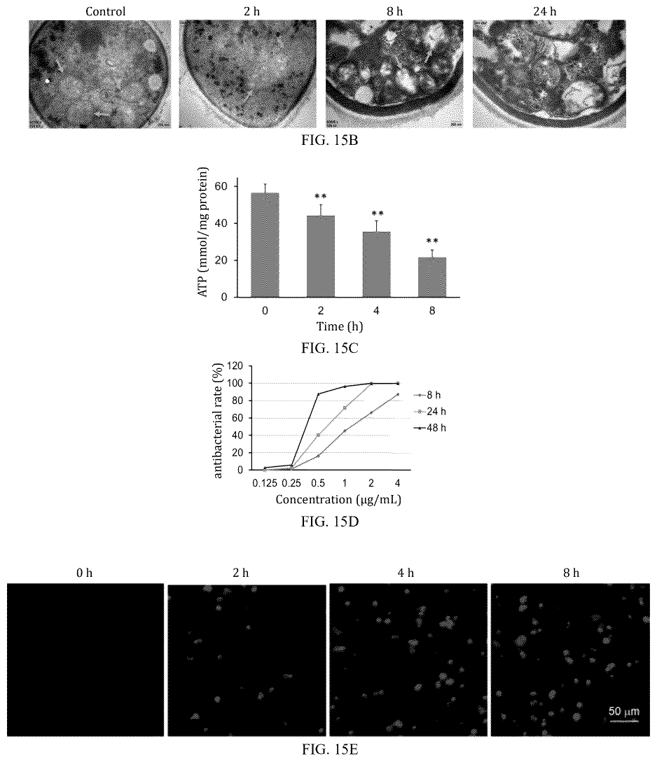

Two molds, Cryptococcus neoformans H99 and Candida albicans ATCC90029, were cultured in a yeast peptone glucose agar medium (YPD) for 24 h at 28° C., respectively; and the diameters of the inhibition zones were recorded. The results were shown in FIG. 15A. It can be seen that the cation of the example in the present application had an excellent anti-mold effect.

To study an anti-fungal mechanism of the cation of the example in the present application, the cell morphology of Cryptococcus neoformans cells was observed under TEM. As shown in FIG. 15B, normal Cryptococcus neoformans cells had intact cell membranes, thick sacs, uniform center density, standard mitochondrial structures, and regular cell morphology. However, in cells treated with the salt of Example 1 at 1.0 g/mL for 8 h, the cell membranes and mitochondria of the cells were damaged. With the extension of the treatment time of the salt of Example 1, the rupture of the cell membranes was more significant, the mitochondria appeared vacuolated, and DNAs were aggregated and entangled together. The results showed that the salt of Example 1 directly damaged Cryptococcus neoformans cells with the concentration increase time.

In addition, after the treatment with the salt of Example 1, the ATP production level of Cryptococcus neoformans decreased significantly (see FIG. 15C), and its apoptotic cells increased over time (see FIG. 15D). The Cryptococcus neoformans cells were fixed with 2% formaldehyde for 30 min, added with 0.1% Triton X-100 and cultured for 30 min at room temperature. Then, an APO-BrdU TUNEL Assay Kit was used to detect apoptosis. The results were shown in FIG. 15E. It can be seen that apoptotic cells increased over time after the treatment with the salt of Example 1.

Therefore, it can be concluded that the anti-fungal mechanism of the cation of the example in the present application may be related to the inhibition of fungal energy supply and the induction of apoptosis.

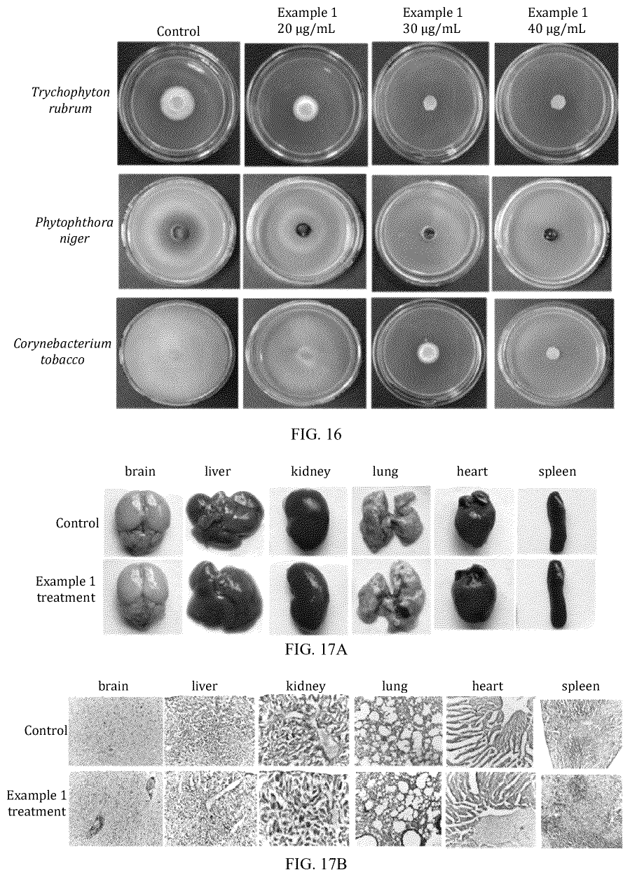

The anti-ascomycetes activity of the salt of Example 1 against three ascomycetes, Trichophyton rubrum ATCC28188, Aspergillus niger, and Corynespora cassiicola, was detected by means of a potato glucose agar (PDA) medium plate method according to the recommendation of CLSI. The results were shown in FIG. 16. It can be seen that the cation of the example in the present application had an excellent anti-ascomycetes effect.

### In Vivo Toxicity Test for Mice

In order to determine whether the cation of the example in the present application can be used in animals, a mouse toxicity test was performed. Mice were randomly divided into two groups: a control group and an Example 1 treatment group. The mice in the Example 1 treatment group were injected with the salt of Example 1 at a total dose of 200 mg/kg (dissolved in normal saline) through the tail veins within 20 min. The mice in the control group were given an equal volume of saline by tail vein injection. After the injection, no significant abnormalities were seen in both groups of mice. Then, the mice were euthanized 24 h after administration of the salt of Example 1, and blood samples and organs (brain, kidney, heart, lung, liver, and spleen) were separated for biochemical testing, respectively. Tissue images and HE staining showed no significant damages or inflammations in any of the tissues (see FIG. 17A, and FIG. 17B). In addition, the results of blood routine examination (using an automated hemocytometer) showed that all the indicators of the mice were within the normal ranges.

### Test for Tumor-Bearing Mice

Tumor-bearing mice were prepared by anesthetizing mice with 3% sodium pentobarbital and then injected subcutaneously with 10⁶ H22 cells diluted with 200 μL of PBS through the axilla. The tumor volume (V) was calculated by a longest diameter (L) and a maximum transverse diameter (W) in a vertical direction of a tumor, which were measured with vernier calipers, according to a formula V=LW²/2. After cell transplantation, the tumor volume increased day by day. When the tumor volume reached about 0.2 cm³, the mice were randomly divided into five groups: a normal saline treatment group (model group), a cisplatin treatment group (positive control group), an Example 1 low-dose treatment group, an Example 1 medium-dose treatment group, and an Example 1 high-dose treatment group. Eight mice were assigned to each group for a total of 40 mice. The mice in respective doses of the Example 1 treatment group were intravenously injected with 2 mg/kg (low dose), 4 mg/kg (medium dose) and 8 mg/kg (high dose) of the salt of Example 1, respectively; the mice in the positive control group were intravenously injected with 8 mg/kg of cisplatin; and the mice in the model group were given the same amount of normal saline once a day for 9 consecutive days. The subcutaneous tumors in each group were observed and recorded until the mice in the model group were euthanized with excessive sodium pentobarbital.

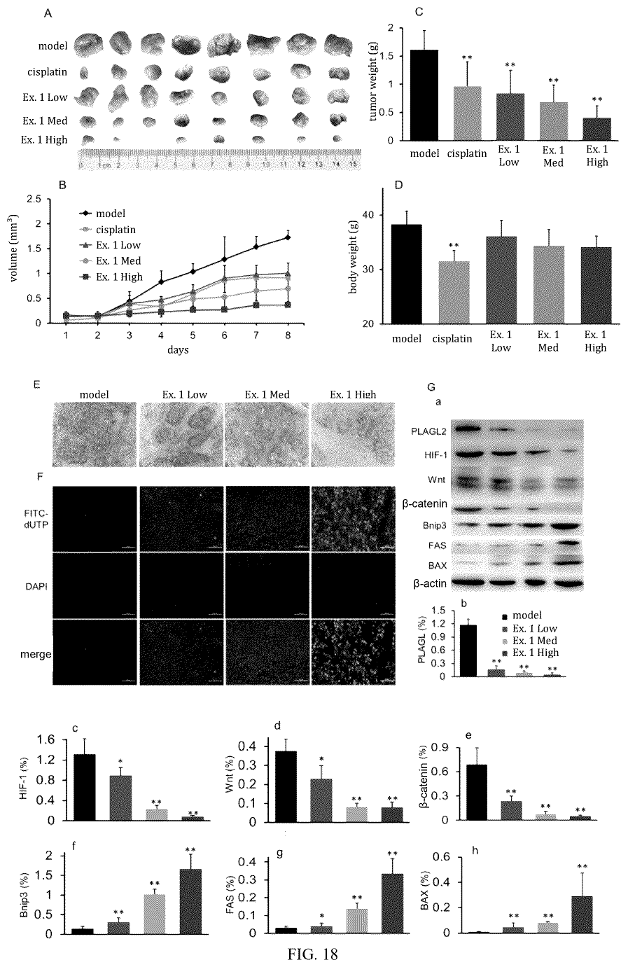

The results were shown in FIG. 18. It can be seen that the tumor volume of each control mouse in the normal saline treatment group was increasing every day. However, tumor progression was delayed in mice treated with the salt of Example 1, and the reduction in tumor volume and weight also increased with increasing doses (see FIG. 18, A-C). The tumor weight in the Example 1 low-dose treatment group was approximately the same as that in the cisplatin treatment group, and the weight of each mouse in the cisplatin treatment group decreased significantly, but there was no significant difference in mouse weight between each dose of the Example 1 treatment group and the control group (see FIG. 18, D).

Tumor tissues were subjected to HE staining, and the results were shown in FIG. 18 E. The tumor tissues of the mice in the control group were filled with dense cell clumps, however, in the mice treated with the salt of Example 1, the area of cell clumps was reduced. The tumor tissues were subjected to TUNEL staining, and the results were shown in FIG. 18 F. A large number of apoptotic cells appeared in the tumor tissues after the treatment with the salt of Example 1, indicating that Example 1 might effectively inhibit tumor cell proliferation by inducing apoptosis in vivo. In addition, as the concentration of the salt of Example 1 increased, more apoptotic cells appeared, indicating that apoptosis was induced by the salt of Example 1 in a dose-dependent pattern. Western blot analysis showed that the salt of Example 1 reduced the levels of PLAGL2, HIF-1, Wnt, and β-catenin, while upregulating the expression of pro-apoptotic proteins BAX, BNIP3, and FAS (FIG. 8G).

### Test for Cryptococcus-Infected Mice

Mice were injected with 0.2 mL of PBS solution of 10⁶ cfu of Cryptococcus neoformans through the tail veins, and a Cryptococcus-infected mouse model (Cryptococcus encephalitis and pneumonia model) was established.

After 8 h of injection, the mice were randomly divided into five groups: a normal saline treatment group (model group), a fluconazole treatment group (positive control group), an Example 1 low-dose treatment group, an Example 1 medium-dose treatment group, and an Example 1 high-dose treatment group. The mice in respective doses of the Example 1 treatment group were intravenously injected with 2 mg/kg (low dose), 4 mg/kg (medium dose) and 8 mg/kg (high dose) of the salt of Example 1, respectively; the mice in the positive control group were intraperitoneally administered with 8 mg/kg of fluconazole; and the mice in the model group were given the same amount of normal saline. Drug treatment was started 4 h after infection once a day for 9 consecutive days. The mice were euthanized 24 h after the last administration, and brains and lungs of the mice were removed in sections for HE and PAS staining, and homogenized for the detection of the loads of fungal tissues.

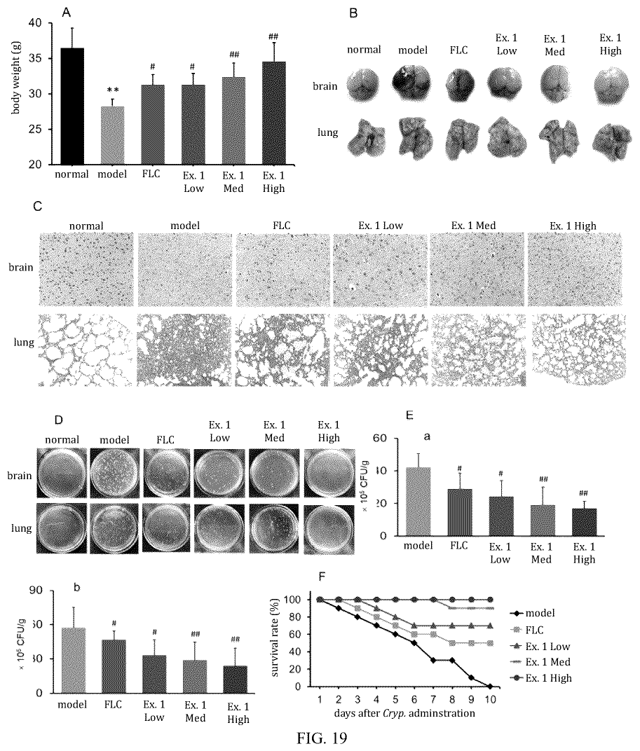

The results were shown in FIG. 19. The mice in the normal saline treatment group showed significant Cryptococcus-infected symptoms, such as fragility, depression, somatic sensation, hypocinesis and weight loss. However, after the treatment with Example 1 and fluconazole, the symptoms were improved, and the weight gained (see FIG. 19, A). Pathological anatomy of the brains and lungs of the mice showed the mice treated with normal presented acute cerebral edema and pneumonia compared to normal mice, while pathological symptoms were improved after the treatment with the salt of Example 1 or fluconazole (see FIG. 19, B). PAS and HE staining also showed the appearance of large numbers of Cryptococcus in the brains and lungs, resulting in a decrease in neurons in the brains, nerve fiber disorders, and lung tissue hyperplasia (see FIG. 19C). After the culture of tissue homogenates, several Cryptococcus grew on a solid medium in the normal saline treatment group (see FIG. 19, D). However, compared with the mice treated with normal saline, the mice treated with the salt of Example 1 had significantly reduced bacteria counts in the brains and lungs, accompanied with attenuated inflammations (see FIGS. 19, C and E). In addition, the number of Cryptococcus in the lungs and brains decreased with increasing doses.

In addition, all mice in the normal saline treatment group died within 10 days of administering Cryptococcus, but the survival rates of various doses of the Example 1 treatment group 1 were 100% (high dose), 90% (medium dose), and 50% (low dose), respectively (see FIG. 19, F), showing excellent anti-Cryptococcus activity in the mice.

Test of Tumors Combined with Fungi Infections

Tumor-bearing mice were prepared by anesthetizing mice with 3% sodium pentobarbital and then injected subcutaneously with 10⁶ H22 cells diluted with 200 μL of PBS through the axilla. When the tumor volume reached about 0.2 cm³, the mice were injected with 0.2 mL of PBS solution of 10⁶ cfu of Cryptococcus neoformans through the tail veins.

Then, the mice were randomly divided into five groups: a normal saline treatment group (model group), a cisplatin and fluconazole treatment group (positive control group), an Example 1 low-dose treatment group, an Example 1 medium-dose treatment group, and an Example 1 high-dose treatment group. The mice in respective doses of the Example 1 treatment group were intravenously injected with 4 mg/kg (low dose), 8 mg/kg (medium dose) and 16 mg/kg (high dose) of the salt of Example 1, respectively; the mice in the positive control group were intraperitoneally administered with 8 mg/kg of fluconazole, and intravenously injected with 8 mg/kg of cisplatin; and the mice in the model group were given the same amount of normal saline.

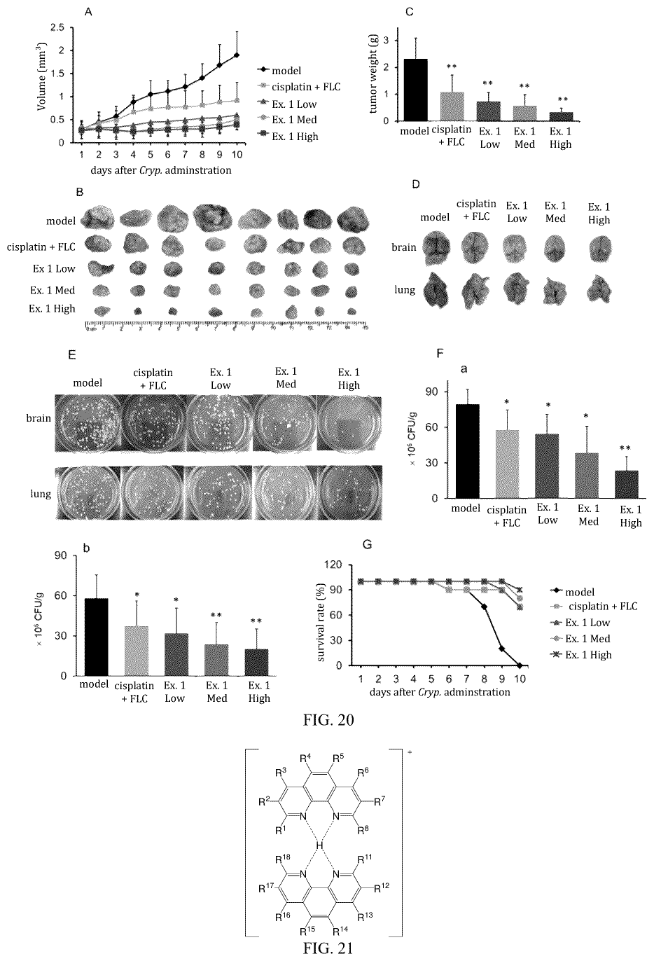

After consecutive administration, the mice were euthanized, and the weights and the fungi count of tumor tissues in the brains and lungs were measured. The results were shown in FIG. 20.

The growth of the tumors was significantly delayed after the treatment with the salt of Example 1 (see FIG. 20, A-C). The symptoms of cerebral edema and pneumonia in the mice were improved at the same time (see FIG. 20, D), and the Cryptococcus counts in the brains and lungs were significantly reduced (see FIGS. 20, E and F); and the survival rate increased from 0 to 70% (low dose), 80% (medium dose), and 90% (high dose), respectively (see FIG. 20, G).

The document disclosed by the inventors of the present application within one year prior to the filing date of the present application (or the priority date of the application claiming the priority of the present application), Zizhen Zhao et al. “Synthesis of Hemiprotonic Phenanthroline-Phenanthroline⁺ Compounds with both anti-tumor and Antimicrobial Activity.” J. Med. Chem. 2022, 65, 2532-2547, was incorporated herein by reference in its entirety.

The above embodiments are only the preferred embodiments of the present application and cannot be intended to limit the protection scope of the present application, and any non-substantial changes and substitutions made by those skilled in the art on the basis of the present application also fall into the protection scope of the present application.

1-10. (canceled)

11. A protonated dimer cation, comprising or consisting of a structure of formula (A) or a resonant structure thereof, wherein “ [symbol] ” in Formula (A) is a covalent bond or a non-covalent bond, 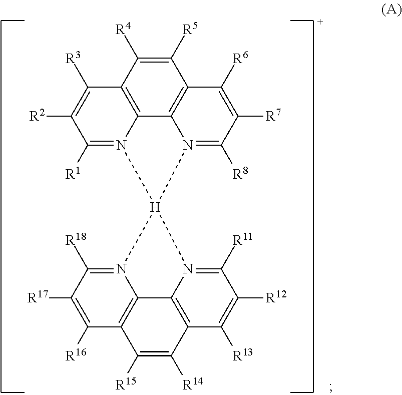
wherein, R¹, R², R³, R⁴, R⁵, R⁶, R⁷, R⁸, R¹¹, R¹², R¹³, R¹⁴, R¹⁵, R¹⁶, R¹⁷, and R¹⁸ are each independently selected from a group consisting of hydrogen, methyl, phenyl, cyano, amino, nitro, and chloro.

12. The cation according to claim 11, wherein
R¹ and R¹¹ are each independently selected from a group consisting of hydrogen, methyl, cyano and chloro;
R², R⁷, R¹², and R¹⁷ are each independently selected from a group consisting of hydrogen and methyl;
R³, R⁶, R¹³, and R¹⁶ are each independently selected from a group consisting of hydrogen, methyl and phenyl;
R⁴ and R¹⁴ are each independently selected from a group consisting of hydrogen, nitro and amino;
R⁵ and R¹⁵ are hydrogen; and
R⁸ and R¹⁸ are each independently selected from a group consisting of hydrogen and methyl.

13. The cation according to claim 11, wherein
R¹, R⁵, R⁸, R¹¹, R¹⁵ and R¹⁸ are hydrogen;
R², R³, R⁶, R⁷, R¹², R¹³, R¹⁶, and R¹⁷ are each independently selected from a group consisting of hydrogen and methyl;
R⁴ and R¹⁴ are each independently selected from a group consisting of hydrogen and amino.

14. The cation according to claim 11, wherein
R¹ and R¹¹ are the same;
R², R⁷, R¹², and R¹⁷ are the same;
R³, R⁶, R¹³, and R¹⁶ are the same;
R⁴ and R¹⁴ are the same;
R⁵ and R¹⁵ are the same; and
R⁸ and R¹⁸ are the same.

15. The cation according to claim 11, wherein R¹, R⁸, R¹¹, and R¹⁸ are hydrogen.

16. The cation according to claim 11, wherein R², R³, R⁶, R⁷, R¹², R¹³, R¹⁶, and R¹⁷ are hydrogen.

17. The cation according to claim 11, wherein R⁴, R⁵, R¹⁴, and R¹⁵ are hydrogen.

18. The cation according to claim 11, wherein
R¹¹, R¹², R¹³, R¹⁴, R¹⁵, R¹⁶, R¹⁷, and R¹⁸ are hydrogen.

19. The cation according to claim 11, wherein
R¹, R², R³, R⁴, R⁵, R⁶, R⁷, R⁸, R¹¹, R¹², R¹³, R¹⁴, R¹⁵, R¹⁶, R¹⁷, and R¹⁸ are hydrogen; or
R¹, R⁸, R¹¹, and R¹⁸ are methyl; and R², R³, R⁴, R⁵, R⁶, R⁷, R¹², R¹³, R¹⁴, R¹⁵, R¹⁶, and R¹⁷ are hydrogen; or
R², R³, R⁶, R⁷, R¹², R¹³, R¹⁶, and R¹⁷ are methyl; and R¹, R⁴, R⁵, R⁸, R¹¹, R¹⁴, R¹⁵, and R¹⁸ are hydrogen; or
R³, R⁶, R¹³, and R¹⁶ are phenyl; and R¹, R², R⁴, R⁵, R⁷, R⁸, R¹¹, R¹², R¹⁴, R¹⁵, R¹⁷, and R¹⁸ are hydrogen; or
R⁴ and R¹⁴ are nitro, and R¹, R², R³, R⁵, R⁶, R⁷, R⁸, R¹¹, R¹², R¹³, R¹⁵, R¹⁶, R¹⁷, and R¹⁸ are hydrogen; or
R⁴ and R¹⁴ are amino, and R¹, R², R³, R⁵, R⁶, R⁷, R⁸, R¹¹, R¹², R¹³, R¹⁵, R¹⁶, R¹⁷, and R¹⁸ are hydrogen; or
R¹, R⁸, R¹¹, and R¹⁸ are chloro; and R², R³, R⁴, R⁵, R⁶, R⁷, R¹², R¹³, R¹⁴, R¹⁵, R¹⁶, and R¹⁷ are hydrogen.

20. The cation according to claim 11, wherein
R¹, R², R³, R⁴, R⁵, R⁶, R⁷, R⁸, R¹¹, R¹², R¹³, R¹⁴, R¹⁵, R¹⁶, R¹⁷, and R¹⁸ are hydrogen.

21. A salt, comprising:
the cation according to claim 11; and
a first anion comprising at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion.

22. The salt according to claim 21, wherein the first anion comprises at least one selected from a group consisting of a hexafluorophosphate ion, a hexafluorosilicate ion, a chloride ion, and a sulfate ion.

23. A method of preparing of the cation according to claim 11, comprising:
contacting a first reactant with a first acid, to obtain a first acid salt of the first reactant;
contacting the first acid salt of the first reactant with a first salt comprising a first anion in a first acidic environment, to obtain a first solution comprising a protonated first acid salt cation of the first reactant and the first anion;
adding a second reactant to the first solution in a second acidic environment, to obtain a second solution or a first intermediate; and
adding a first base to the second solution or contacting the first base with the first intermediate,
wherein the first reactant comprising a compound of formula (C), and the second reactant comprising a compound of formula (D),

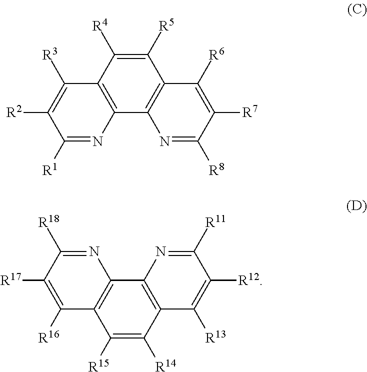

24. The method according to claim 23, wherein
the first acid is selected from a group consisting of hydrochloric acid, hydrobromic acid, and hydroiodic acid; and
the first salt is consisting of a first cation and the first anion, and the first cation is an ammonium ion;
and/or
the first reactant is contacted with the first acid at a first temperature, and the first temperature is 0° C. to 70° C.;
the first acid salt of the first reactant is contacted with the first salt at a second temperature, and the second temperature is 0° C. to 70° C.; and
the second reactant is added to the first solution at a third temperature, and the third temperature is 0° C. to 70° C.;
and/or a molar ratio of the first reactant to the first acid is 1:2 to 1:10;
a molar ratio of the first acid salt of the first reactant to the first salt is 1:2 to 1:10; and
a molar ratio of a protonated first reactant cation to the second reactant is 1:1 to 1:2;
and/or
the first acidic environment refers to that the pH is less than or equal to 6; and
the second acidic environment refers to that the pH is less than or equal to 6;
and/or
the first base comprises at least one selected from a group consisting of ammonia water, sodium hydroxide, potassium hydroxide, and triethanolamine; and
the first base is added to the second solution or contacted with the first intermediate to adjust the pH of the second solution or a solution comprising the first intermediate to be greater than 8.

25. A method for controlling or inhibiting microbe on or in a subject or treating a tumor in the subject, comprising
administering the cation of claim 21 to the subject.

26. The method according to claim 25 for controlling or inhibiting the microbe on or in the subject, wherein the microbe is bacteria and/or fungi.

27. The method according to claim 26, wherein the bacteria are selected from a group consisting of Staphylococcus aureus, Staphylococcus epidermidis, hemolytic staphylococcus, Enterococcus faecalis, Streptococcus mutans, Streptococcus thermophilus, Streptococcus lactis, Streptococcus bovis, Corynebacterium diphtheriae, Listeria monocytogenes, Bacillus subtilis, Bacillus cereus, Clostridium perfringens, Escherichia coli, Salmonella enteritidis, Klebsiella pneumoniae, Proteus vulgaris, Yersinia enterocolitis, Pseudomonas aeruginosa, and Pseudomonas fluorescens; and

28. The method according to claim 26, wherein the fungi are selected from a group consisting of Cryptococcus neoformans, Candida albicans, Trichophyton rubrum, Malassezia globosa, Malassezia furfur, Aspergillus flavus, and Aspergillus niger.

29. The method according to claim 25 for treating a tumor in the subject.

30. The method according to claim 29, wherein the tumor is selected from a group consisting of liver cancer, gastric cancer, colon cancer, bladder cancer, breast cancer, pancreatic cancer, prostate cancer, ovarian cancer, neuroblastoma, glioma, and melanoma.
